(19) United States
(12) Patent Application Publication
(10) Pub. No.: US 2024/0238282 A1
(43) Pub. Date: Jul. 18, 2024

(54) METHODS OF TREATING CANCER AND ISCHEMIA DISEASES BY INHIBITION AND INTERVENTION OF ATR PROLYL ISOMERIZATION

(71) Applicant: The University of Toledo, Toledo, OH (US)

(72) Inventor: Yue Zou, Toledo, OH (US)

(73) Assignee: The University of Toledo, Toledo, OH (US)

(21) Appl. No.: 18/557,651

(22) PCT Filed: Apr. 26, 2022
(86) PCT No.: PCT/US2022/026334

§ 371 Date: Oct. 27, 2023

(51) Int. Cl. A61K 31/496; A01K 67/0278; A61K 31/282; A61K 31/4745; A61K 31/513; A61K 31/519; A61K 31/7068; A61P 35/00

(52) U.S. Cl. CPC A61K 31/496; A01K 67/0278; A61K 31/282; A61K 31/4745; A61K 31/513; A61K 31/519; A61K 31/7068; A61P 35/00; A01K 2217/052; A01K 2227/105

(57) ABSTRACT

Combination treatments for cancers involving inhibiting cis-ATR, as well as biomarkers for cancer diagnosis and prognosis, and transgenic animals, are described.

### RELATED APPLICATIONS

This application claims priority to U.S. Provisional Application No. 63/180,843, filed under 35 U.S.C. § 111(b) on Apr. 28, 2021, the disclosure of which is incorporated herein by reference in its entirety for all purposes.

### STATEMENT REGARDING FEDERALLY SPONSORED RESEARCH

This invention was made with government support under Grant Number CA219342 awarded by the National Institutes of Health. The government has certain rights in this invention.

### SEQUENCE LISTING

The instant application contains a Sequence Listing which has been submitted electronically in ASCII format and is hereby incorporated by reference in its entirety. Said ASCII copy, created on Apr. 26, 2022, is named 62679-WO-PCT_SL.txt and is 4,718 bytes in size.

### BACKGROUND

Cell death and immortality are closely associated with cancer and cancer therapeutics, as oncogenesis requires a compromise of apoptosis while most cancer therapies depend on apoptosis. Precise regulation of programmed cell death (apoptosis) is essential for cellular and organ homeostasis. Hyperactive apoptosis is associated with human diseases such as myocardial infarction, ischemic stroke, and immunodeficiency. In contrast, dysfunctional apoptosis is highly relevant to oncogenesis and, also, cancer treatment. While the mechanisms regulating apoptotic pathways are extensively investigated and well defined, there is limited progress in understanding how antiapoptotic pathways protect cells. While apoptotic signaling leads to initiation of apoptosis, eventual execution requires disabling antiapoptotic machineries. Thus, pro-apoptotic and antiapoptotic pathway coordination is necessary for execution of apoptosis. Unveiling new mechanisms involved in regulating apoptosis would be significant in disease prevention, diagnosis, and treatment. In addition, the underlying mechanisms as to how silent oncogenic mutations become active during aging remain elusive and defining the mechanisms requires a reliable aging-dependent oncogenesis model. There remains a need in the art for new and improved cancer treatments.

### SUMMARY

Provided is a method for treating a cancer comprising administering to a subject having a cancer an effective amount of a PP2A inhibitor to inhibit cis-ATR together with a cancer therapeutic drug to treat the cancer. In certain embodiments, the PP2A inhibitor is LB-100. In certain embodiments, the cancer therapeutic drug comprises a chemotherapeutic agent, an immunotherapeutic agent, or a hormonal therapeutic agent. In certain embodiments, the PP2A inhibitor and the cancer therapeutic drug are administered simultaneously. In certain embodiments, the PP2A inhibitor and the cancer therapeutic drug are administered sequentially. In certain embodiments, the PP2A inhibitor is administered alone to treat DNA damaging drug resistant cancer.

Further provided is a method for treating a cancer comprising administering to a subject having a cancer an effective amount of a DAPK1 inhibitor to inhibit cellular cis-ATR together with a cancer therapeutic drug to treat the cancer. In certain embodiments, the DAPK1 inhibitor is HS38. In certain embodiments, the cancer therapeutic drug comprises a chemotherapeutic agent, an immunotherapeutic agent, or a hormonal therapeutic agent. In certain embodiments, the DAPK1 inhibitor and the cancer therapeutic drug are administered simultaneously. In certain embodiments, the DAPK1 inhibitor and the cancer therapeutic drug are administered sequentially.

Further provided is a method for treating a cancer comprising administering to a subject having a cancer an effective amount of a Pin1 agonist to inhibit cis-ATR together with a cancer therapeutic drug to treat the cancer. In certain embodiments, the cancer therapeutic drug comprises a chemotherapeutic agent, an immunotherapeutic agent, or a hormonal therapeutic agent. In certain embodiments, the Pin1 agonist and the cancer therapeutic drug are administered simultaneously. In certain embodiments, the Pin1 agonist and the cancer therapeutic drug are administered sequentially.

Further provided is a method for treating a cancer comprising administering to a subject having a cancer an effective amount of two or more of a PP2A inhibitor, a DAPK1 inhibitor, and a Pin1 agonist to inhibit cellular cis-ATR level together with a cancer therapeutic drug to treat the cancer. In certain embodiments, the PP2A inhibitor is LB-100. In certain embodiments, the DAPK1 inhibitor is HS38. In certain embodiments, the cancer therapeutic drug comprises a chemotherapeutic agent, an immunotherapeutic agent, or a hormonal therapeutic agent. In certain embodiments, the PP2A inhibitor, a DAPK1 inhibitor, and a Pin1 agonist are administered simultaneously with the cancer therapeutic drug. In certain embodiments, the PP2A inhibitor, a DAPK1 inhibitor, and a Pin1 agonist are administered sequentially with the cancer therapeutic drug.

Further provided is a method for treating a cancer comprising administering to a subject having a cancer an effective amount of a cis-ATR inhibitor together with a cancer therapeutic drug to treat the cancer. In certain embodiments, the cis-ATR inhibitor is a PP2A inhibitor, a DAPK1 inhibitor, or a Pin1 agonist. In certain embodiments, the cancer therapeutic drug comprises a chemotherapeutic agent, an immunotherapeutic agent, or a hormonal therapeutic agent.

Further provided is a method for treating a cancer comprising administering to a subject having a cancer an effective amount of a cis-ATR inhibitor alone to treat a DNA damaging drug resistant cancer. In certain embodiments, the cis-ATR inhibitor is a PP2A inhibitor, a DAPK1 inhibitor, or a Pin1 agonist.

Further provided is a method of diagnosing a cancer or making a prognosis, the method comprising measuring cis-ATR level or activity in cells, cell cytoplasm, or tissues of a human subject, and diagnosing the human subject as having a cancer, or making a prognosis, based on the measured cis-ATR level or activity.

Further provided is a method of diagnosing a tumor, the method comprising measuring an amount of dephosphorylation of cytoplasmic ATR-S431 in a tissue of a subject, and diagnosing a tumor in the subject based on the measured amount of dephosphorylation.

Further provided is the use of a cis-ATR level or activity in cells, cell cytoplasm, or tissues of a human subject as a biomarker for cancer diagnosis or prognosis.

Further provided is the use of dephosphorylation of cytoplasmic ATR-S431 as a tumor biomarker.

Further provided is a pharmaceutical composition comprising a cis-ATR inhibitor and one or more cancer therapeutic drugs. In certain embodiments, the cis-ATR inhibitor is a PP2A inhibitor, a DAPK1 inhibitor, or a Pin1 agonist. In certain embodiments, the cancer therapeutic drugs comprise chemotherapeutic agents, immunotherapeutic agents, or hormonal therapeutic agents.

Further provided is a kit comprising a first container housing cis-ATR inhibitor; and a second container housing a cancer therapeutic drug. In certain embodiments, the cis-ATR inhibitor is a PP2A inhibitor, a DAPK1 inhibitor, or a Pin1 agonist. In certain embodiments, the kit further comprises a pharmaceutically acceptable carrier, diluent, or excipient.

Further provided is a transgenic animal comprising a C57BL/6 mouse having a single amino acid substitution of Ser431 of ATR with alanine, wherein the single amino acid substitution silences phosphorylation of ATR-S431 required to isomerize cis-ATR to trans-ATR.

Further provided is a transgenic animal comprising a C57BL/6 mouse having a single amino acid substitution of Pro432 of ATR with alanine, wherein the single amino acid substitution sterically locks ATR in its trans-isomeric form throughout cells in the mouse.

In some embodiments of any method, composition, or use described herein, the cancer therapeutic drug is selected from the group consisting of: erlotinib, docetaxel, fluorouracil, 5-fluorouracil, gemcitabine, PD-0325901, cisplatin, carboplatin, paclitaxel, temozolomide, tamoxifen, doxorubicin, Akti-1/2, HPPD, rapamycin, lapatinib, oxaliplatin, bortezomib, sutent, letrozole, imatinib mesylate, XL-518, ARRY-886, SF-1126, BEZ-235, XL-147, ABT-869, ABT-263, PTK787/ZK 222584, fulvestrant, leucovorin (folinic acid), lonafamib, sorafenib, gefitinib, irinotecan, tipifamib, capecitabine, abraxane, albumin-engineered nanoparticle formulations of paclitaxel, vandetanib, chloranmbucil, AG1478, AG1571, temsirolimus, pazopanib, canfosfamide, thioTepa and cyclosphosphamide, bullatacin, bullatacinone, bryostatin, callystatin, CC-1065 or analogs thereof, cryptophycin 1, cryptophycin 8, dolastatin, duocarmycin or analogs thereof, leutherobin, pancratistatin, sarcodictyin, spongistatin, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimnustine, clodronate, esperamicin, neocarzinostatin chromophore and related chromoprotein enediyne antibiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin, methotrexate, denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex, razoxane, rhizoxin, sizofuran, spirogermanium, tenuazonic acid, triaziquone, 2,2′,2″-trichlorotriethylamine, T-2 toxin, verracurin A, roridin A, anguidine, urethane, vindesine, dacarbazine, mannomustine, mitobronitol, mitolactol, pipobroman, gacytosine, arabinoside, cyclophosphamide, thioTepa, 6-thioguanine, mercaptopurine, vinblastine, etoposide, ifosfamide, mitoxantrone, vincristine, vinorelbine, novantrone, teniposide, edatrexate, daunomycin, aminopterin, ibandronate, CPT-11, topoisomerase inhibitor RFS 2000, difluoromethylomithine (DMFO), paclitaxel, abraxane (paclitaxel albumin-stabilized nanoparticle formulation), afinitor, erlotinib hydrochloride, everolimus, gemcitabine hydrochloride, oxaliplatin (eloxatin), capecitabine, cisplatin, irinotecan, colinic acid, folfox, folfirinox, nab-paclitaxel with gemcitabine, metformin, digoxin, simvastatin, nivolumab, pembrolizumab, rituximab, durvalumab, cemiplimab, anastrozole, exemestane, letrozole, tamoxifen, raloxifene, fulvestrant, toremifene, gosrelin, leuprolide, triptorelin, apalutamide, enzalutamide, darolutamide, bicalutamide, flutamide, nilutamide, abiraterone, ketoconazole, degarelix, medroxyprogesterone acetate, megestrol acetate, mitotane, and combinations thereof.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIGS. 1A-1G: Transgenic knock-in ATR^{P432A} (trans-ATR) and ATR^{S431A} (cis-ATR) mice: ATR^{P432A/P432A} is embryonically lethal. FIG. 1A shows DNA sequence configurations for CRISPR-Cas9-generated transgenic knock-in mice at the Pin1 isomerization motif (Ser431Pro432) of ATR. The transgenic mice contain a single residue substitution of S or P with alanine. FIG. 1A discloses SEQ ID NOS 12-14, respectively, in order of appearance. FIG. 1B shows the transgenic mice were genotyped by restriction cleavage analysis with enzymes Afe1 and Sfo1, specifically for ATR^{P432A} and ATR^{S431A} genotypes, respectively. The uncut PCR full-length fragment is 459 bp long and cleaved mutant allele fragments are 204 bp and 255 bp long, respectively. FIG. 1C shows Sanger DNA sequencing confirms ATR genotypes, Base ambiguities are indicated as W=A or T, K=T or G, R=A or G, S=C or G, and M=A or C. FIG. 1C discloses SEQ ID NOS 12, 15, 13, 16-20, 12 and 15, respectively, in order of appearance. FIG. 1D shows WB analysis (3-8% gradient SDS-Page) confirms the presence of trans- and cis-ATR in the cytoplasm of ATR^{P432A} and ATR^{S431A} mouse liver cells, respectively. Nuclear ATR is the trans-ATR regardless of genotypes (gel-loading ratio of nuclear to cytoplasm is 1:3). The UV-induced cis-ATR formation in human cells serves as a control. FIG. 1E shows representative H&E staining showing the gastrulation of normal and abnormal E7.5/8.5 embryos. AMN: amnion; ExE: extraembryonic ectoderm; ExEC: ExE cavity. FIG. 1F shows a summary of ATR^{+/S431A} or ATR^{+/P432A} mouse breeding studies. FIG. 1G shows genotypes of E13.5 embryos from ATR^{+/P432A} mouse breeding, indicating the embryonic lethality of ATR^{P432A/P432A} homozygotes.

FIGS. 2A-2I: Cis-ATR is an oncogenic protein promoting aging-dependent spontaneous oncogenesis in vivo. FIG. 2A shows lymphoma in an aged cis-ATR (ATR^{+/S431A} or ATR^{S431A/S431A}) mouse. The photo shows an enlarged, inflamed mesenteric lymph node tumor together with representative H&E staining images from FFPE sections of the tumor (8× represents 8-time magnification of original images). Representative immunohistochemical (IHC) stains show high expressions of Ki-67 (proliferation marker), CD5, and CD3e (T-cell lymphoma markers) in the lymph node tumor as compared to the WT spleen. However, there is little or no difference between the tumor and WT spleen in staining for CD20, a B-cell marker. FIG. 2B shows TCR and BCR repertoire analyses by PCR of V(D)J junctions reveal that the lymphomas have a likely T cell clonal lineage. Comparison of Vγ TCR repertoires among the lymphoma tissues (L) and WT spleen (C), or among lymphoma tissues (L), blood of mice with lymphoma (B), and blood from WT mice (C) reveals significant differences. In contrast, rearranged VH=1 genes in lymphoma tissues (L) show no difference from WT spleen tissue (C) in BCR rearrangements. FIG. 2C shows three top-left photos showing WT liver and two representative enlarged, tumor-laden livers from cis-ATR mice. The representative H&E images of formalin-fixed liver tissue sections show a lymphocytic infiltration near a central vein (yellow arrow), multiple giant cells, nuclear atypia, and mitotic figures (red arrows). Representative IHC stains show positive staining of alpha-fetoprotein (AFP, a liver cancer marker) and CD3e, but negative staining of CD20. FIG. 2D shows the summary of spontaneous cancer incidences of specific mouse genotypes in two aging groups. *p<0.05 by Fisher-exact test for comparison of ATR^{+/S431A} and ATR^{+/S431A/S431A} to ATR_{+/+} or ATR^{+/S431A} (analysis indicates that there is no significant difference between ATR^{+/S431A} and ATR^{+/S431A/S431A} mice. (%): Enlarged Peyer's patches indicative of increased systemic inflammation. FIG. 2E shows a duolink PILA analysis showing the binding of ATR to pro-apoptotoic mitochondrial/cytoplasmic tBid in liver cancer FFPE tissue sections of ATR^{+/S431A} mice vs. a normal mouse liver. The plot shows the fluorescent PLA signal per 1,000 cells from three different tissue areas in mean±SD, using CellProfiler Analyst. FIG. 2F shows IHC staining of phosphorylated ATR(S431) in mouse WT liver vs, tumor-containing liver from a cis-ATR (ATR^{+/S431A}) mouse. The phosphorylation occurs specifically in the cytoplasm. Quantification was performed for p-ATR(S431) IHC intensity in the cytoplasm vs nucleus per 1,000 cells using CellProfiler Analyst. FIG. 2G shows IHC staining of p-ATR(S431), Ki-67, ATR, and CD3e in cis-ATR liver tissue tumors showing that the tissue areas deficient in p-ATR(S431) are the regions proliferating most (Ki-67), but having little T cell infiltration. FIG. 2H shows a WE analysis of age-dependent DNA damage and the induced checkpoint signaling in the livers of WT mice aged 4, 12, and 20 months from 3 separate experiments: M1, M2, M3. FIG. 2I shows tissues of WT mice at different ages collected and subjected to lysis and cell fractionation. The obtained cytoplasmic fractions were analyzed by WB for the levels of p-ATR(S431).

FIGS. 3A-3E: Analysis of the antiapoptotic function of cis-ATR in human cancer via TCGA data mining. FIG. 3A shows a schematic illustration of the regulation of ATR isomerization pathway by Pin1, DAPK1, and PP2A proteins, and their influences on cis-ATR formation in the cytoplasm. FIG. 3B shows a Wilcoxon paired test on the effects of the expression level of Pin1, PP2A, and DAPK1, or Subgroup B versus Subgroup A on 5-year survival probability of all 17 cancer types in humans. FIG. 3C shows a plot showing the relative 5-year survival probability of subgroup B versus subgroup A in each type of cancer where each data point represents a type of cancer. FIG. 3D shows a cancer type-independent analysis of unpaired 5-year survival probability between subgroup B and A. FIG. 3E shows patient-derived pancreatic cancer PDCL5 cells treated with PDAC therapy drug gemcitabine (1.0 uM), FDA-approved PP2A inhibitor LB-100 (5 uM), or the combination for 72 hours. FIGS. 3F-3G show two patient-derived pancreatic cancer cells, PDCL5 and PDCL15, subjected to MTT assays after treating with the PDAC therapy drug gemcitabine (1.0 μM or 0.3 μM, respectively) and PP2A inhibitor LB-100 (5 μM), alone or in combination, for 72 hours. PANC-1 cells were treated with gemcitabine (1.0 μM) or FOLFIRINOX (5-FU, 10 μM, oxaliplatin, 0.1 μM, leucovorin, 0.15 μM, irinotecan, 0.1 μM), alone or in combination with LB-100 (1.0 μM), for 72 hours before the MTT assay. FIG. 3H shows PLA assays performed to measure cis-ATR-tBid interactions at mitochondria in cells treated as described in FIG. 3G. FIG. 3I shows MTT assays: acquired gemcitabine-resistance pancreatic cancer G3K cells which were generated from the parental MiaPaCa-2 cells with stepwise treatment of gemcitabine up to 3 μM, were treated with PP2A inhibitor LB-100 at the indicated concentrations in the absence of gemcitabine. FIG. 3J shows PLA assays conducted to detect the association of cis-ATR with tBid at mitochondria in the treated cells.

FIGS. 4A-4F: Cis-ATR preponderance inhibits apoptosis but has no effect on ATR-dependent DNA damage checkpoint signaling in human cells, FIG. 4A shows transgenic A375 melanoma ATR^{S429A/S428A} (S428A), ATR^{P429A/P429A} (P429A), and ATR^{+/+} (WT) cell lines subjected to MTT assays following UV (60 J/m²), followed by a 24 hour recovery, or CPT and carboplatin (1.35 μM) for 16 and 24 hours, respectively. FIG. 4B shows WB analysis of UV- or CPT-induced apoptosis activation in the transgenic melanoma cells (FIG. 4A) as evidenced by the cleavages of caspases 3 and 7. FIG. 4C shows WB analysis of ATR-dependent DNA damage and checkpoint signaling in response to UV irradiation in the A375 transgenic melanoma cells. FIG. 4D shows a similar UV-induced DNA damage and checkpoint signaling analysis performed on ATR^{flox/-} HCT116 cells transfected with ATR-WT, ATR-S428A or ATR-P429A expression constructs. FIG. 4E shows an illustration of the biological consequences of the off-balance levels of trans- and cis-ATR in the cytoplasm. It should be noted that the normal balance of trans- and cis-ATR proteins in WT cells does not mean that the two isoforms are in the same amount in the cytoplasm. In fact, in unstressed normal cells, trans-ATR overwhelmingly dominates over cis-ATR. The diagram is a representation of the normal in-balance ratio of the two isoforms in WT cells. FIG. 4F shows an illustration of the mechanisms in which cis-ATR plays a role in aging-dependent oncogenesis, cancer therapeutic resistance, and poor cancer prognosis. Δp-ATR^{S428} stands for dephosphorylation of p-ATR^{S428}.

FIG. 5: Mechanisms and pathway of ATR isomerization in cells.

FIG. 6: Breeding of WT ATR (ATR^{+/+}), ATR^{+/S431A} and ATR^{+/P432A} mice demonstrates normal Mendelian distribution of litter sizes with no significant differences in the viviparity between WT mice and mutant mice with a single residue substitution.

FIG. 7: Relative changes of blood lymphocyte count of WT and cis-ATR mice during aging. Changes of mouse blood lymphocyte count with increasing age were measured relative to the average counts of a study group of WT mice with normal counts (range: 3.40×10⁹-7.44×10⁹) at the 1st month (Month 1) of the study. All mice in the three groups (WT, cis-ATR without tumors, and cis-ATR mice with tumors) had the lymphocyte counts within the normal range when the counting study started. The first-month ages of the mice are equivalent among the three groups of mice.

FIGS. 8A-8C: Cis-ATR promotes aging-dependent spontaneous oncogenesis of melanoma in vivo. FIG. 8A shows the multiple, melanated lesions on skin and seminal vesicle. Representative H&E images (FIG. 8B) show the multiple atypical cells in cis-ATR mouse skin, and the representative IHC staining of formalin-fixed skin sections (FIG. 8C) reveals an increased staining for Ki-67 proliferation and melon A, a melanoma marker, in cis-ATR mouse skin sections.

FIGS. 9A-9C: Cis-ATR promotes aging-dependent spontaneous oncogenesis of colon cancer in vivo. FIG. 9A shows a photo of a mouse's inflamed and distended colon by distal tumor. FIG. 9B shows representative H&E images of sections of formalin-fixed tissue, showing a distorted mucosa in the cis-ATR mouse colon tumor, with immune cell infiltration. FIG. 9C shows representative IHC staining showing the higher expression of carcinoembryonic antigen (CEA) and widespread expression of Ki-67 in the colon tumor formed in cis-ATR mice as compared to WT mouse colon tissues.

FIG. 10: IHC staining of the phosphorylation of ATR-S431, p-ATR(S431), in heterozygous cis-ATR (ATR^{+/S431A}) mouse lymphoma vs. WT spleen tissues. The phosphorylation occurs predominately in the cytoplasm of WT mouse spleen tissues as compared to a marked reduction in p-ATR(S431) in a cis-ATR mouse lymphoma. Quantification was performed for p-ATR(S431) IHC staining in the cytoplasm versus nucleus per 100 cells of WT spleen tissues, and in the cytoplasm of WT spleen versus cis-ATR mouse lymphoma tissues, per 100 cells using CellProfiler Analyst 2.2.1.

FIG. 11: Univariate and multivariate Cox regression analyses of 5-year survival on high vs low mRNA expression levels of Pin1, PP2A, and DAPK1, or Subgroup B vs. Subgroup A in 17 cancer types.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

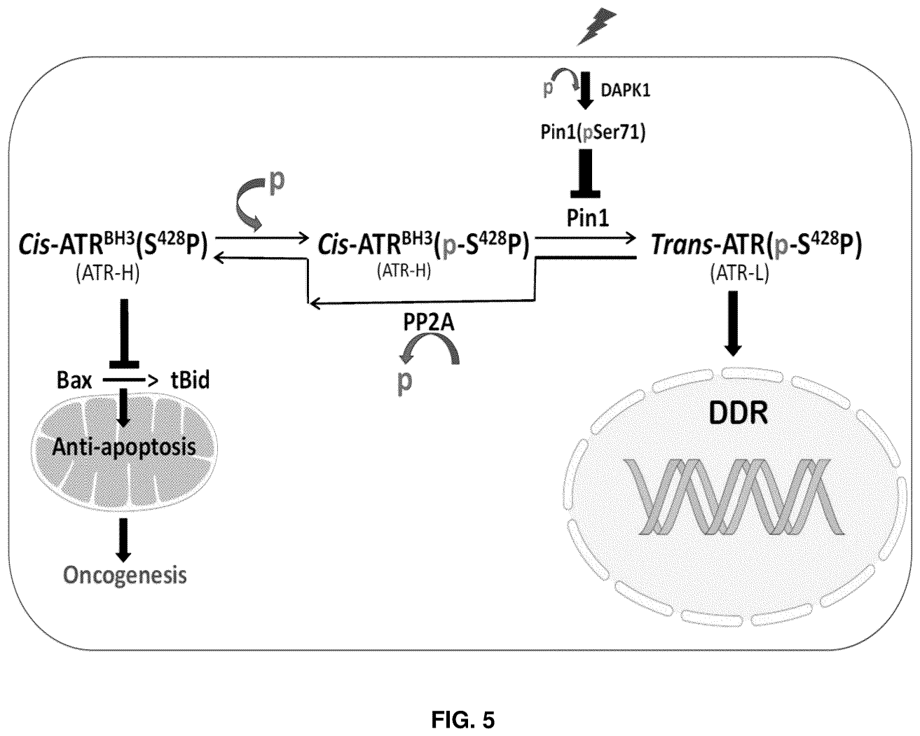

Human ataxia telangiectasia and Rad3-related (ATR), a member of the phosphatidylinositol 3-kinase-related kinase (PIKK) family, plays a crucial role in maintaining genome integrity during DNA damage responses (DDR). While ATR-dependent DDR checkpoint signaling is the major function of ATR and occurs in the nucleus, ATR also plays an important antiapoptotic role at mitochondria to prevent cell death in a kinase activity-independent manner (FIG. 5). This antiapoptotic activity depends on cytoplasmic formation of the prolyl cis-isomer of ATR (cis-ATR, ATR-H) against trans-ATR (ATR-L) at S428P429 motif. This ATR isomeric balance is regulated by Pin1 prolyl isomerase, which converts cis-ATR to trans-ATR. DNA damage promotes cytoplasmic cis-ATR formation via inhibiting Pin1 and dephosphorylating pS428-ATR, although nuclear ATR always remains in the trans-ATR form.

ATR forms prolyl cis/trans isomers in the cytoplasm via isomerization at Ser428Pro429 motif. Cis-ATR is antiapoptotic at mitochondria. It is demonstrated in the examples herein that ATR is a molecular switch for determining the cell fate between cell death and immortality using transgenic knock-in ATR-S431A and ATR-P432A mice (human ATR-S428A and ATR-P429A). The presence of cis-ATR as an oncogenic protein in the cytoplasm is important for oncogenesis to occur and cancer cells to survive. In addition, cellular cis-ATR level increases during normal aging and dramatically high levels of cis-ATR are found in tumors. Analysis of PP2A, DAPK1, and Pin1 proteins that regulate cis-ATR in 48,000 human cancer cases of 17 types shows a strong inverse correlation between cis-ATR and cancer patient survival. Thus, cancer combination therapies using inhibitors or agonists of cis-ATR regulating proteins PP2A, DAPK1, and Pin1, together with other cancer therapeutic drugs, are provided herein. Since most cancer therapies critically depend on apoptosis, this strategy of combination therapy can be used to sensitize various cancer therapeutic drugs to overcome cancer resistance in cancer treatments. Furthermore, in accordance with the present disclosure, cis-ATR is established as a biomarker for cancer diagnosis and prognosis.

In one aspect, a PP2A inhibitor, such as LB-100, can be used to inhibit cis-ATR together with a cancer therapeutic drug as a combination therapy to treat cancer and cancer resistance. PP2A is a serine/threonine phosphatase implicated in diverse cellular processes. LB-100 is a small molecule inhibitor of PP2A having a formula of C₁₃H₂₀N₂O₄ and the following structure:

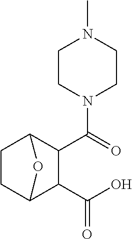 [SMILES: CN1CCN(C(=O)C2C3CCC(O3)C2C(=O)O)CC1]

However, other PP2A inhibitors are possible and encompassed within the scope of the present disclosure.

In another aspect, a DAPK1 inhibitor can be used to inhibit cellular cis-ATR together with a cancer therapeutic drug as a combination therapy to treat cancer and cancer resistance. A non-limiting example DAPK1 inhibitor is the small molecule HS38, which has the following structure:

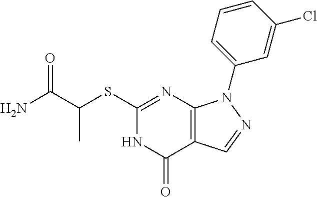 [SMILES: CC(Sc1nc2c(cnn2-c2cccc(Cl)c2)c(=O)[nH]1)C(N)=O]

However, other DAPK1 inhibitors are possible and encompassed within the scope of the present disclosure.

In another aspect, a Pin1 agonist (rather than inhibitor, as commonly proposed in the literature) can be used to inhibit cis-ATR together with a cancer therapeutic drug as a combination therapy to treat cancer and cancer resistance.

In another aspect, two or more of a PP2A inhibitor, a DAPK1 inhibitor, and a Pin1 agonist can be used to inhibit cellular cis-ATR level together with a cancer therapeutic drug as a combination to treat cancer or cancer resistance.

In another aspect, any cis-ATR inhibitor (including a PP2A inhibitor, a DAPK1 inhibitor, or a Pin1 agonist) can be used in combination with a cancer therapeutic drug as a combination therapy to treat cancer or cancer resistance.

In another aspect, the cis-ATR level or activity in cells, cell cytoplasm, or tissues of humans can be used as a biomarker for cancer diagnosis and prognosis.

As described herein, a cancer therapeutic drug may be any chemotherapeutic agent. Suitable chemotherapeutic agents include, but are not limited to: taxane compounds, such as paclitaxel; platinum coordination compounds; topoisomerase I inhibitors, such as camptothecin compounds; topoisomerase II inhibitors, such as anti-tumor podophyllotoxin derivatives; anti-tumor vinca alkaloids; anti-tumor nucleoside derivatives; alkylating agents; anti-tumor anthracycline derivatives; HER2 antibodies; estrogen receptor antagonists or selective estrogen receptor modulators; aromatase inhibitors; differentiating agents, such as retinoids, and retinoic acid metabolism blocking agents (RAMBA); DNA methyl transferase inhibitors; kinase inhibitors; farnesyltransferase inhibitors; HDAC inhibitors, or other inhibitors of the ubiquitin-proteasome pathway; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide, and trimethylomelamine; acetogenins; camptothecins, such as the synthetic analog topotecan; cryptophycins; nitrogen mustards, such as chlorambucil; nitrosoureas; bisphosphonates; mitomycins; epothilones; maytansinoids; trichothecenes; retinoids, such as retinoic acid; pharmaceutically acceptable salts, acids and derivatives of any of the above; and combinations thereof. Non-limiting examples of specific chemotherapeutic agents include erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethyl-ethanamine, NOLVADEX®, ISTUBAL®, VALODEX®), doxorubicin (ADRIAMYCIN®), Akti-1/2, HPPD, rapamycin, lapatinib (TYKERB®, Glaxo SmithKline), oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (MEK inhibitor, Exelixis, WO 2007/044515), ARRY-886 (MEK inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), ABT-869 (multi-targeted inhibitor of VEGF and PDGF family receptor tyrosine kinases, Abbott Laboratories and Genentech), ABT-263 (Bcl-2/Bcl-xL inhibitor, Abbott Laboratories and Genentech), PTK787/ZK 222584 (Novartis), fulvestrant (FASLODEX®, AstraZeneca), leucovorin (folinic acid), lonafamib (SARASAR™ SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifamib (ZARNESTRA™, Johnson & Johnson), capecitabine (XELODA®, Roche), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thioTepa and cyclosphosphamide (CYTOXAN®, NEOSAR®), bullatacin, bullatacinone, bryostatin, callystatin, CC-1065 (including its adozelesin, carzelesin and bizelesin synthetic analogs), cryptophycin 1, cryptophycin 8, dolastatin, duocarmycin (including the synthetic analogs, KW-2189 and CB1-TM1), leutherobin, pancratistatin, sarcodictyin, spongistatin, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimnustine, clodronate, esperamicin, neocarzinostatin chromophore and related chromoprotein enediyne antibiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, Oreg.), razoxane, rhizoxin, sizofuran, spirogermanium, tenuazonic acid, triaziquone, 2,2′,2″-trichlorotriethylamine, T-2 toxin, verracurin A, roridin A, anguidine, urethane, vindesine, dacarbazine, mannomustine, mitobronitol, mitolactol, pipobroman, gacytosine, arabinoside (“Ara-C”), cyclophosphamide, thioTepa, 6-thioguanine, mercaptopurine, vinblastine, etoposide (VP-16), ifosfamide, mitoxantrone, vincristine, vinorelbine (NAVELBINE®), novantrone, teniposide, edatrexate, daunomycin, aminopterin, ibandronate, CPT-11, topoisomerase inhibitor RFS 2000, and difluoromethylomithine (DMFO), paclitaxel, 5-fluorouracil, abraxane (paclitaxel albumin-stabilized nanoparticle formulation), afinitor (everolimus), erlotinib hydrochloride, everolimus, gemcitabine hydrochloride, oxaliplatin (eloxatin), capecitabine (xeloda), cisplatin, irinotecan (camptosar), colinic acid (leucovorin), folfox (folinic acid, 5-fluorouracil, and oxaliplatin), folfirinox (folinic acid, 5-fluorouracil, irinotecan, and oxaliplatin), nab-paclitaxel with gemcitabine, metformin, digoxin, and simvastatin.

Cancer therapeutic drugs may also include immunotherapeutic agents. Non-limiting examples of immunotherapeutic agents include nivolumab, pembrolizumab, rituximab, durvalumab, cemiplimab, and combinations thereof.

Cancer therapeutic drugs may also include hormonal therapeutic agents. Non-limiting examples of hormonal therapeutic agents include anastrozole, exemestane, letrozole, tamoxifen, raloxifene, fulvestrant, toremifene, gosrelin, leuprolide, triptorelin, apalutamide, enzalutamide, darolutamide, bicalutamide, flutamide, nilutamide, abiraterone, ketoconazole, degarelix, medroxyprogesterone acetate, megestrol acetate, mitotane, and combinations thereof.

Pharmaceutical compositions of the present disclosure may comprise an effective amount of a cis-ATR inhibitor (such as a PP2A inhibitor, a DAPK1 inhibitor, or a Pin1 agonist), optionally with additional agents (such as a cancer therapeutic drug), dissolved or dispersed in a pharmaceutically acceptable carrier, optionally with an additional cancer therapeutic drug. The preparation of a pharmaceutical composition that contains at least one compound or additional active ingredient will be known to those of skill in the art in light of the present disclosure, as exemplified by Remington's Pharmaceutical Sciences, 2003, incorporated herein by reference. Moreover, for animal (e.g., human) administration, it is understood that preparations should meet sterility, pyrogenicity, general safety, and purity standards as required by FDA Office of Biological Standards.

A composition disclosed herein may comprise different types of carriers depending on whether it is to be administered in solid, liquid or aerosol form, and whether it need to be sterile for such routes of administration as injection. Compositions disclosed herein can be administered intravenously, intradermally, transdermally, intrathecally, intraarterially, intraperitoneally, intranasally, intravaginally, intrarectally, intraosseously, periprosthetically, topically, intramuscularly, subcutaneously, mucosally, intraosseosly, periprosthetically, in utero, orally, topically, locally, via inhalation (e.g., aerosol inhalation), by injection, by infusion, by continuous infusion, by localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 2003, incorporated herein by reference).

The actual dosage amount of a composition disclosed herein administered to an animal or human patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a composition herein and/or additional agent is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsules, they may be compressed into tablets, or they may be incorporated directly with the food of the diet.

In further embodiments, a composition described herein may be administered via a parenteral route. As used herein, the term “parenteral” includes routes that bypass the alimentary tract. Specifically, the pharmaceutical compositions disclosed herein may be administered, for example but not limited to, intravenously, intradermally, intramuscularly, intraarterially, intrathecally, subcutaneous, or intraperitoneally (U.S. Pat. Nos. 6,753,514, 6,613,308, 5,466,468, 5,543,158; 5,641,515, and 5,399,363 are each specifically incorporated herein by reference in their entirety).

Solutions of the compositions disclosed herein as free bases or pharmacologically acceptable salts may be prepared in water suitably mixed with a surfactant, such as hydroxypropylcellulose. Dispersions may also be prepared in glycerol, liquid polyethylene glycols and mixtures thereof, and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In some cases, the form must be sterile and must be fluid to the extent that easy injectability exists. It should be stable under the conditions of manufacture and storage and should be preserved against the contaminating action of microorganisms, such as bacteria and fungi. The carrier can be a solvent or dispersion medium containing, for example, water, ethanol, polyol (i.e., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and antifungal agents, such as, but not limited to, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption such as, for example, aluminum monostearate or gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Sterile injectable solutions are prepared by incorporating the compositions in the required amount in the appropriate solvent with various other ingredients enumerated above, as required, followed by filtered sterilization. Generally, dispersions are prepared by incorporating the various sterilized compositions into a sterile vehicle which contains the basic dispersion medium and the required other ingredients from those enumerated above. In the case of sterile powders for the preparation of sterile injectable solutions, some methods of preparation are vacuum-drying and freeze-drying techniques which yield a powder of the active ingredient plus any additional desired ingredient from a previously sterile-filtered solution thereof. A powdered composition is combined with a liquid carrier such as, but not limited to, water or a saline solution, with or without a stabilizing agent.

In other embodiments, the compositions may be formulated for administration via various miscellaneous routes, for example, topical (i.e., transdermal) administration, mucosal administration (intranasal, vaginal, etc.) and/or via inhalation.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones, and luarocapram. Possible bases for compositions for topical application include polyethylene glycol, lanolin, cold cream, and petrolatum, as well as any other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a “patch.” For example, the patch may supply one or more compositions at a predetermined rate and in a continuous manner over a fixed period of time.

In certain embodiments, the compositions may be delivered by eye drops, intranasal sprays, inhalation, and/or other aerosol delivery vehicles. Methods for delivering compositions directly to the lungs via nasal aerosol sprays has been described in U.S. Pat. Nos. 5,756,353 and 5,804,212 (each specifically incorporated herein by reference in their entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts and could be employed to deliver the compositions described herein. Likewise, transmucosal drug delivery in the form of a polytetrafluoroetheylene support matrix is described in U.S. Pat. No. 5,780,045 (specifically incorporated herein by reference in its entirety), and could be employed to deliver the compositions described herein.

It is further envisioned the compositions disclosed herein may be delivered via an aerosol. The term aerosol refers to a colloidal system of finely divided solid or liquid particles dispersed in a liquefied or pressurized gas propellant. The typical aerosol for inhalation consists of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight, and the severity and response of the symptoms.

In particular embodiments, the compounds and compositions described herein are useful for treating cancers or cancer resistance. As described herein, the compounds and compositions herein can be used in combination therapies. That is, the compounds and compositions can be administered concurrently with, prior to, or subsequent to one or more other desired therapeutic or medical procedures or drugs. The particular combination of therapies and procedures in the combination regimen will take into account compatibility of the therapies and/or procedures and the desired therapeutic effect to be achieved. Combination therapies include sequential, simultaneous, and separate administration of the active compound in a way that the therapeutic effects of the first administered procedure or drug is not entirely disappeared when the subsequent procedure or drug is administered.

It is further envisioned that the compounds and methods described herein can be embodied in the form of a kit or kits. A non-limiting example of such a kit is a kit comprising a cis-ATR inhibitor (such as a PP2A inhibitor, a DAPK1 inhibitor, or a Pin1 agonist) and a cancer therapeutic drug in separate containers, where the containers may or may not be present in a combined configuration. Many other kits are possible, such as kits further comprising a pharmaceutically acceptable carrier, diluent, or excipient. The kits may further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive or CD-ROM. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

### Examples

### ATR Isomerization is a Molecular Switch for Cell Death Versus Immortality

Cell death and immortality are associated with numerous human diseases and fatalities, including cancer and ischemia. It has previously been reported that human ataxia telangiectasia and Rad3-related (ATR) forms prolyl cis/trans isomers in the cytoplasm via isomerization at Ser428Pro429 motif1. Cis-ATR, with an active BH3 domain, is antiapoptotic at mitochondria, independent of ATR's kinase activity. In these examples, it is shown that the cytoplasmic isomeric status of ATR can act like a “molecular switch” to determine cell fate between death and immortality. Transgenic mice with a single-residue substitution of ATRP^{432A/P432A} (human ATRP^{429A/P429A}), locking ATR in a trans-ATR isoform (cis-ATRU^{null}), are embryonically lethal. In sharp contrast, ATR^{S431A/S431A} or ATR^{+/S431A} mice, with cis-ATR dominant in the cytoplasm, grew spontaneous tumors during aging. Remarkably, normal mice accumulate cis-ATR during aging. Analysis of human cancer pathological data supports cis-ATR-mediated tumorigenesis. Strikingly, cis- or trans-ATR cells maintain intact normal ATR-dependent DNA damage checkpoint activities. These results reveal an essential role of cis-ATR versus trans-ATR in cell survival, cis-ATR as an oncogenic protein, a unique mechanism of aging-dependent oncogenesis, and the importance of maintaining a delicate balance between cis- and trans-ATR for cellular homeostasis. These findings have implications for therapeutic interventions in cancer and ischemia treatments via manipulating cis-/trans-ATR balance.

### Establishment of Transgenic Knock-In Cis-ATR and Trans-ATR Mouse Models

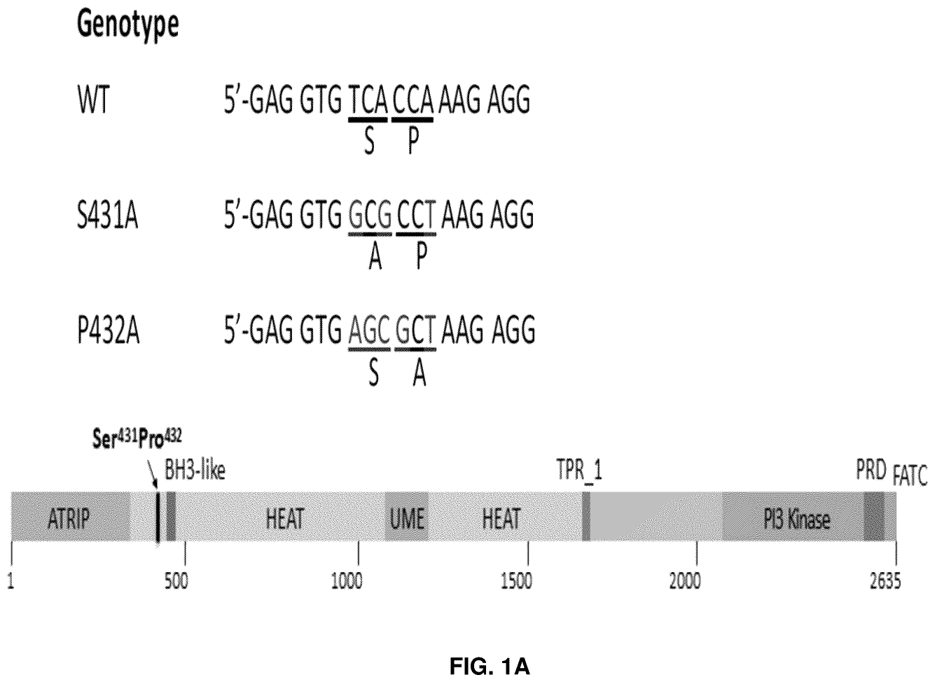

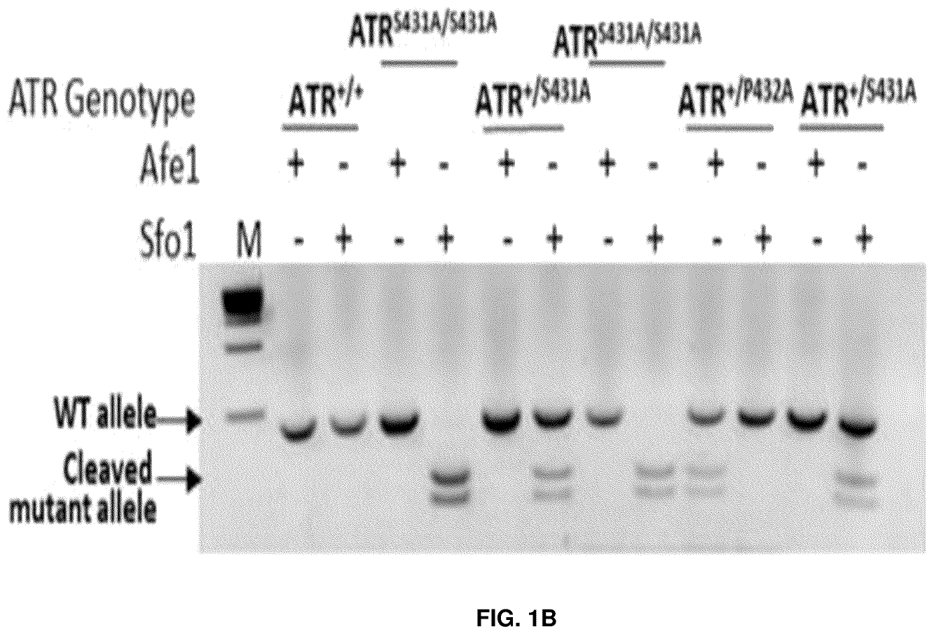

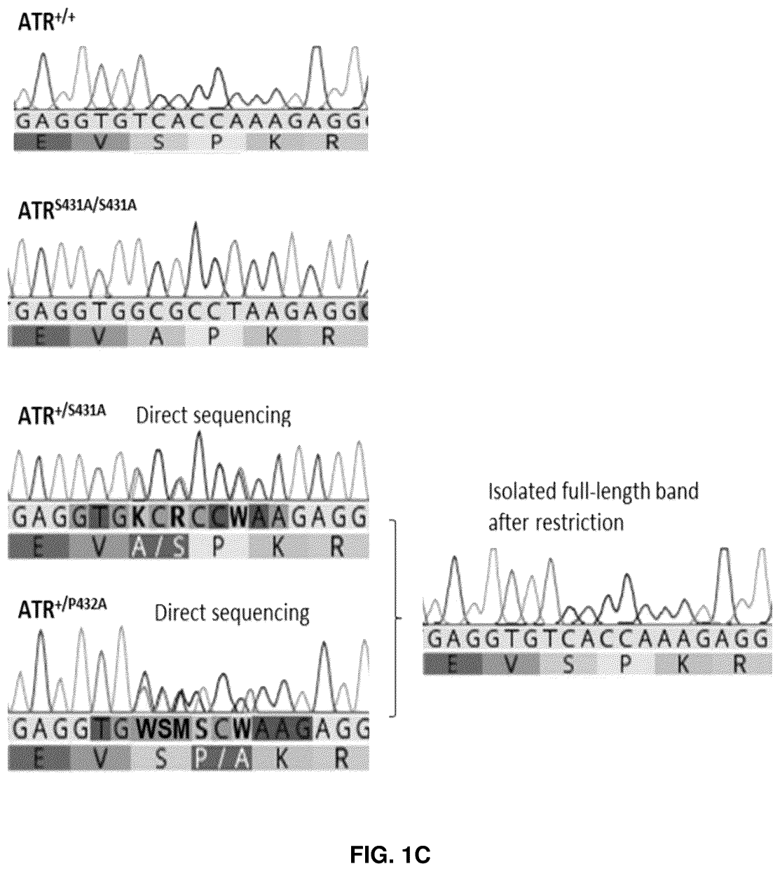

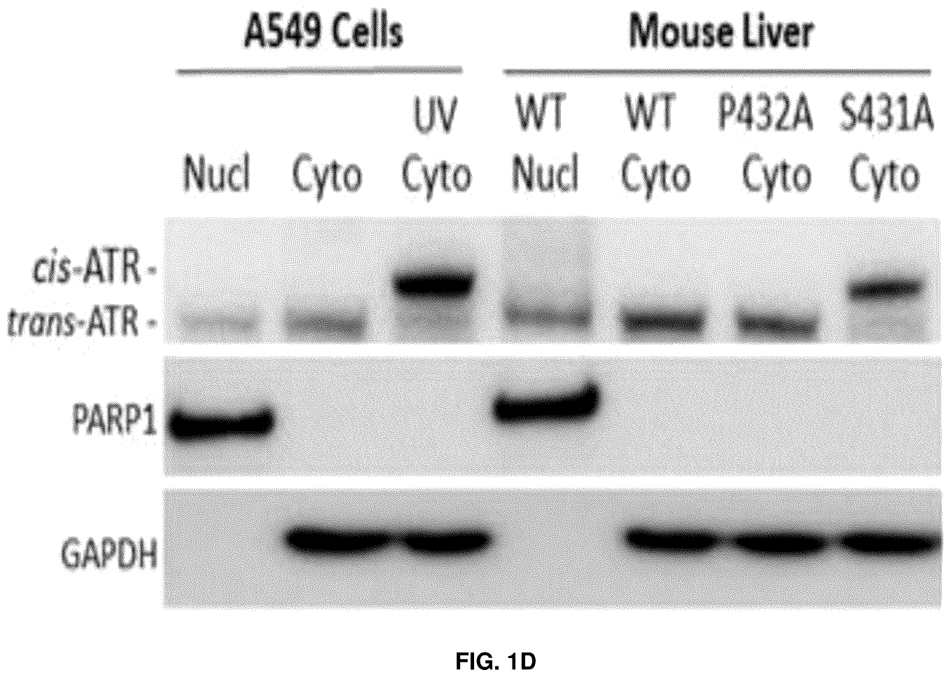

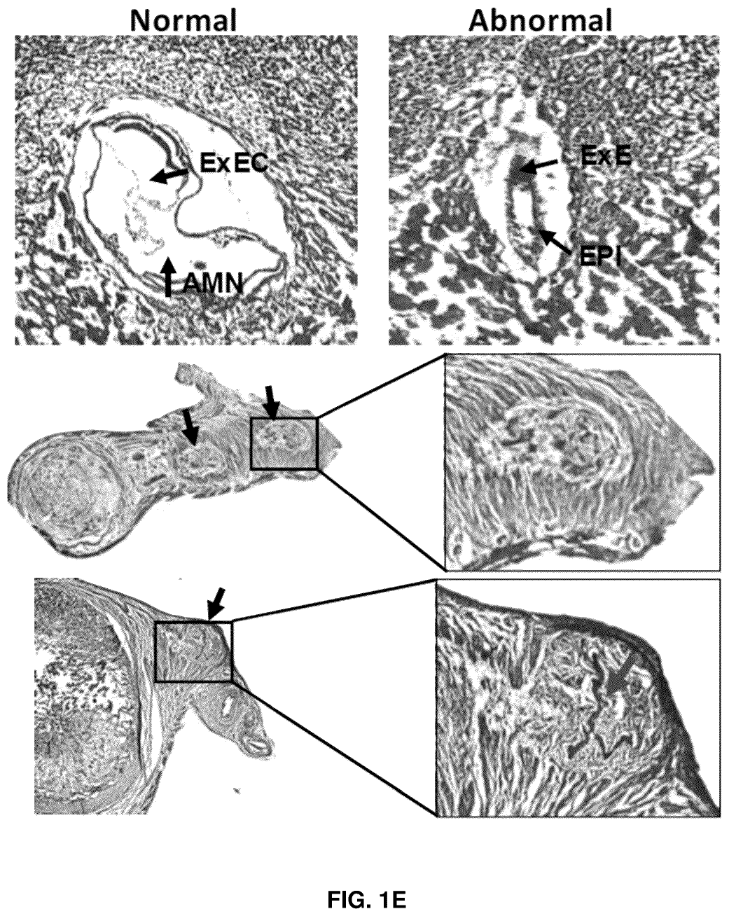

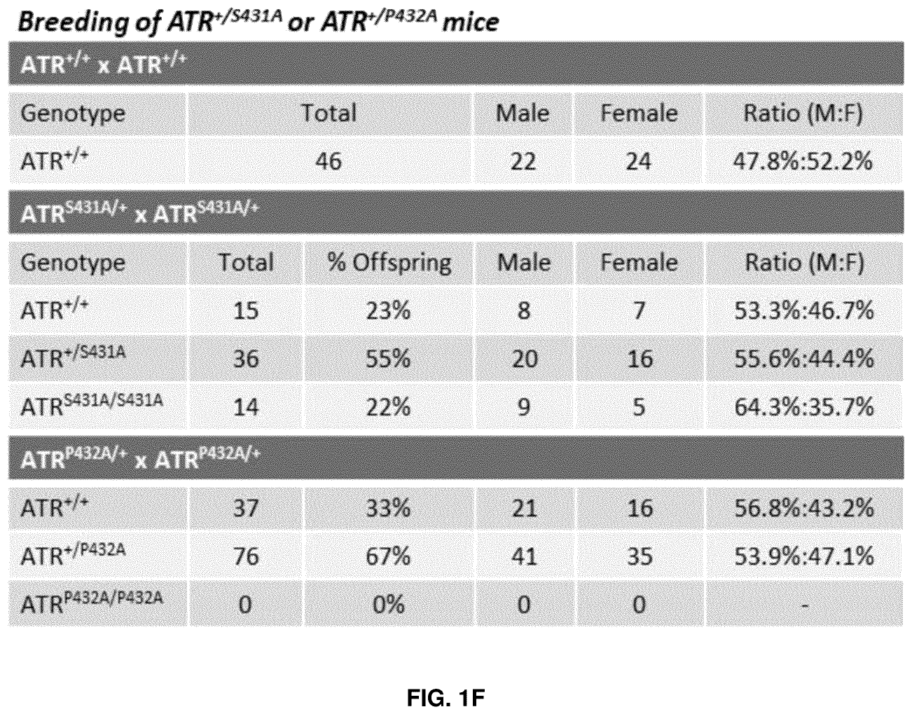

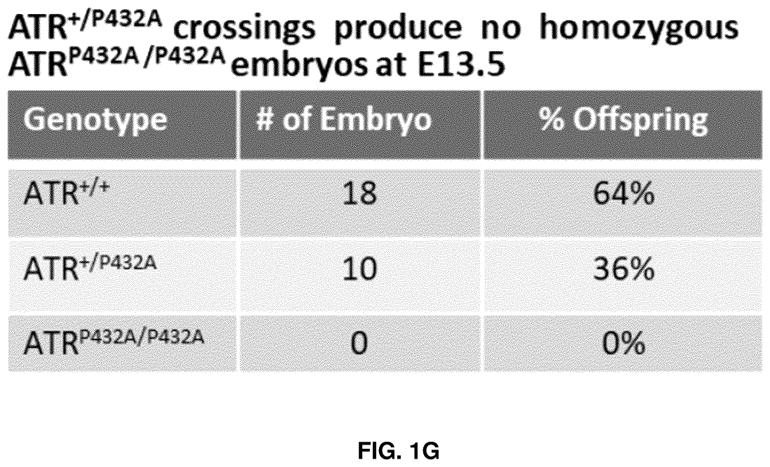

To understand the physio-/pathological significance of cis- and trans-ATR isomers, CRISPR-Cas9 gene editing was used to generate transgenic knock-in mice with a single amino acid substitution of Ser431 or Pro432 of ATR with alanine, ATR^{S431A}, or ATR^{P432A} (homologous to human ATR^{S428A} or ATR^{P429A}, respectively) (FIG. 1A). The ATR^{S431A} substitution silences the phosphorylation of ATR-S431 required by Pin1 to isomerize cis-ATR to trans-ATR. This mutation results in cis-ATR as the dominant ATR prolyl isomeric form in the cytoplasm (nuclear ATR is always trans-). In contrast, the ATR^{P432A} substitution sterically locks ATR in its trans-isomeric form (trans-ATR) throughout the cell as all standard non-proline amino acids are naturally stable in the trans isoform. Therefore, ATR^{S431A} and ATR^{P432A} are cis- and trans-ATR mimics, respectively (for convenience, cis-ATR and trans-ATR will be indicated as ATR^{S431A} and ATR^{P432A}, respectively). While a single amino acid was substituted in the S431P432 motif of ATR, it is the steric structure induced by the substitution that matters to ATR's functions, rather than the substituted residue itself as evidenced below. Mice were genotyped by PCR amplification of the transgenic region, followed by differential restriction digestions (FIG. 1B) and DNA sequencing (FIG. 1C). Expression of cis-ATR or trans-ATR proteins in the corresponding transgenic mice was confirmed by western blotting (WB) of tissue samples (FIG. 1D), which indicates that cis-ATR is the predominant isomeric form in the cytoplasm of ATR^{S431A} mice, while trans-ATR is the only isomeric form in both nucleus and cytoplasm of ATR^{P432A} mice.

### Homozygous ATR^{P432A/P432A} (Trans-ATR) Mice are Embryonically Lethal

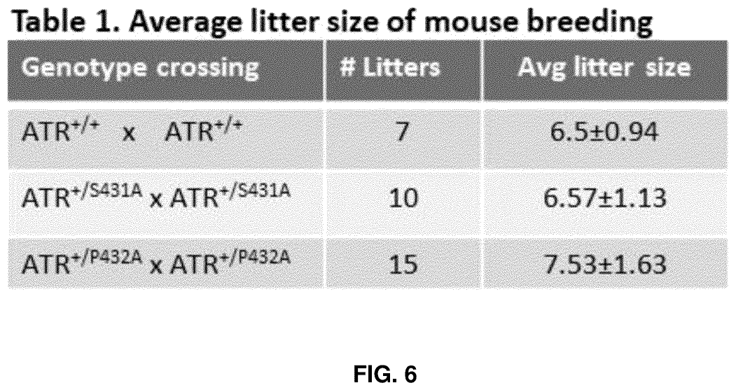

Throughout breeding of trans mouse colonies, no offspring had the homozygous ATR^{P432A/P432A} genotype. To determine if this was a breeding error or if the ATR^{P432A/P432A} genotype was embryonically lethal, a formal breeding study was established. The results show that there was no statistical difference in litter size between the different mouse lines (FIG. 6). Cis-ATR (ATR^{S431A/+}) crossings exhibited normal Mendelian distribution of offspring genotypes (1:2:1=ATR^{+/+}:ATR^{S431A/+}:ATR^{S431A/S431A}). In contrast, trans-ATR (ATR^{P432A/+}) crossings exhibited a non-Mendelian distribution (1:2:0=ATR^{+/+}:ATR^{P432A/+}:ATRP^{432A/P432A}). The absence of homozygous ATR-P432A offspring (FIG. 1F) indicates a striking developmental effect and an embryonic lethality for ATR^{P432A/P432A} mice (cis-ATRnull). Timed pregnancy studies were performed. At E7.5, normal embryos develop into late stage of gastrulation (FIG. 1E, top) displaying amnion (AMN), amnion cavity, extraembryonic ectoderm (ExE) cavity (ExEC), and three intact germ layers, whereas abnormal embryos display no similar structures (FIG. 1E, top right panel). These results show that some embryos have undergone partial reabsorption (middle panel) while full reabsorption at the uterine horn occurred in others (bottom panel).

To confirm that no ATR^{P432A/P432A} embryos were present, 28 embryos were harvested at E13.5 and genotyped (FIG. 1G). While 10 embryos displayed ATR^{+/P432A} and 18 had ATR^{+/+} genotypes, none had the ATR^{P432A/P432A} genotype. This indicates that the ATR^{P432A/P432A} genotype is peri-implantationally lethal (E4.5-7.5). This also explains why litter sizes did not differ substantially across genotypes (FIG. 6), as these embryos likely die and are reabsorbed prior to or shortly after implantation, allowing normal embryos to implant in their place. Notably, cells of ATR^{P432A/P432A} embryos are cis-ATR^{null}. This observed embryonically lethality indicates that either trans-ATR was proapoptotic/anti-growth/anti-survival, or cis-ATR, as an antiapoptotic protein, is required for embryonic survival (FIG. 1D). The former is unlikely as mouse fertility actually depends on ATR kinase activity which is carried out by trans-ATR, mainly in the nucleus. The latter is likely true as the embryonic lethality may be due to the lack of the kinase-independent antiapoptotic activity of cis-ATR, which makes embryonic cells vulnerable to apoptosis, rather than the presence of trans-ATR.

### Cis-ATR Drives Oncogenesis in Mice During Aging

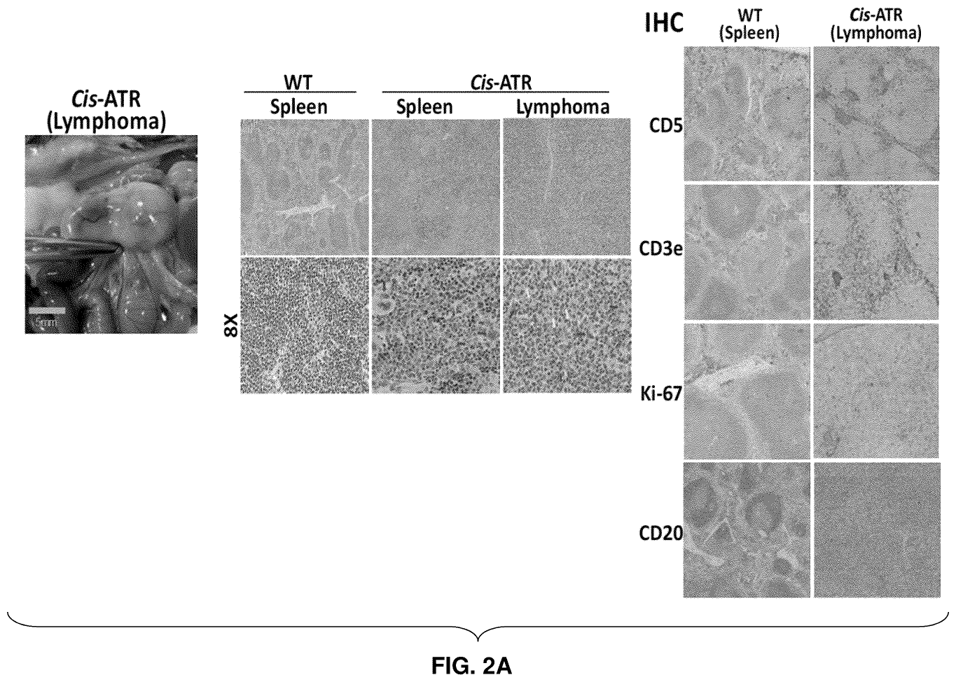

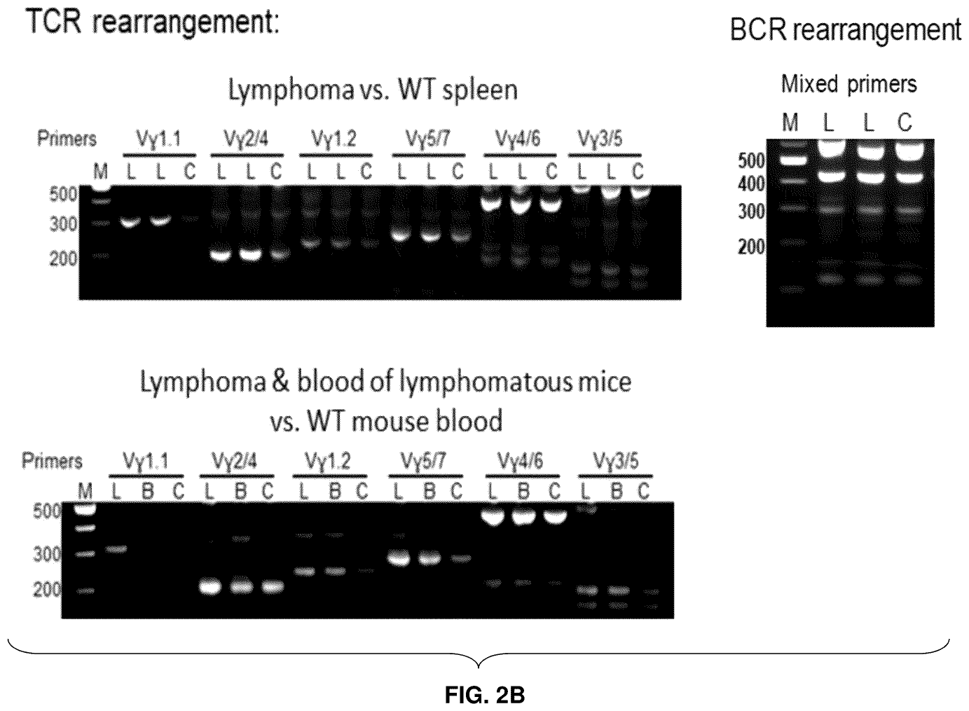

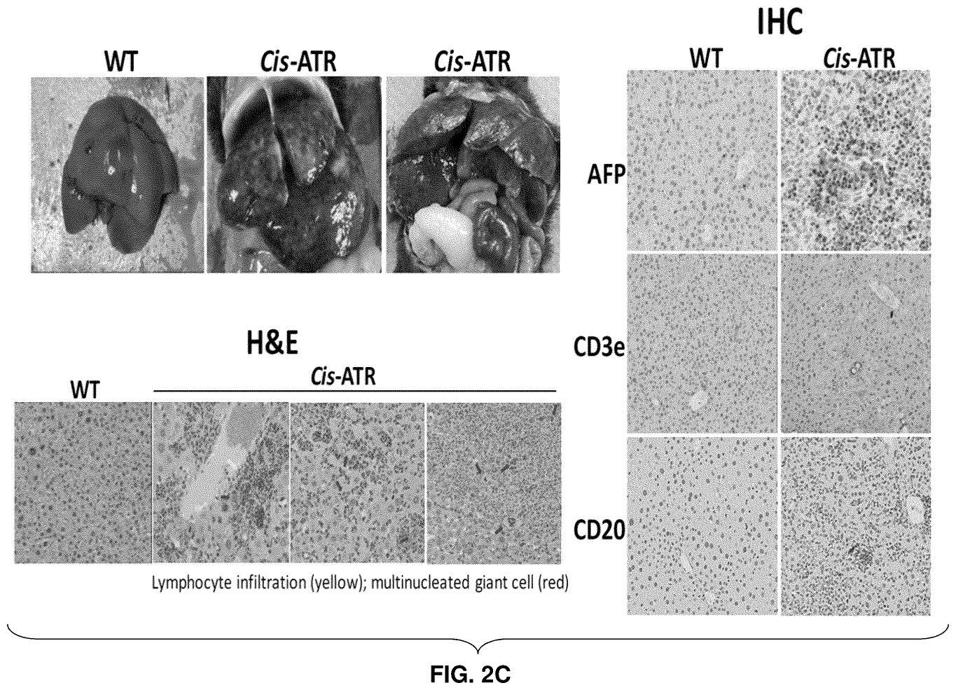

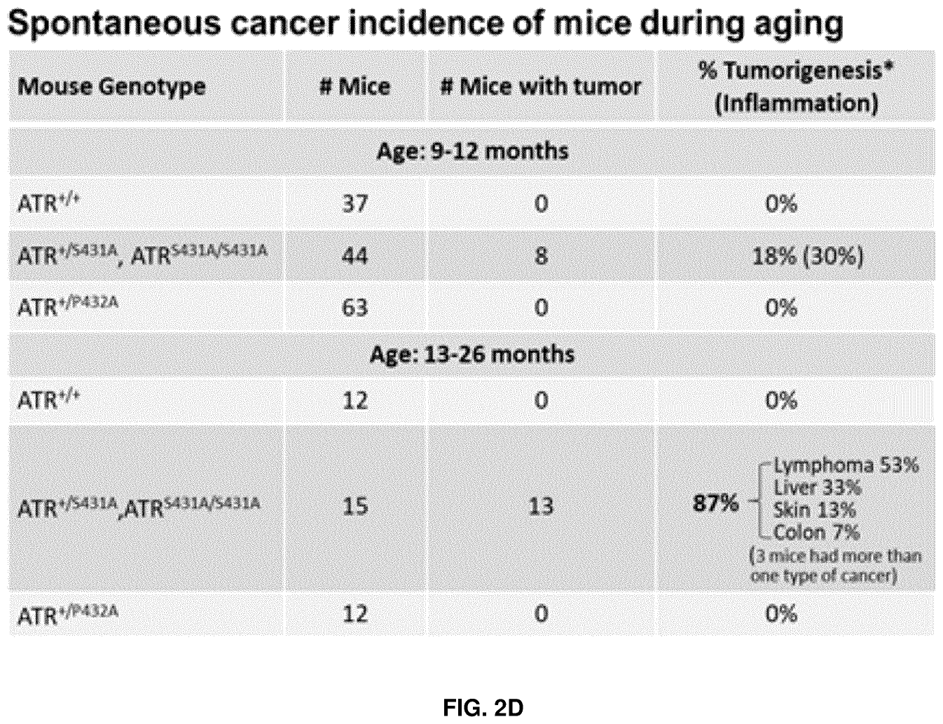

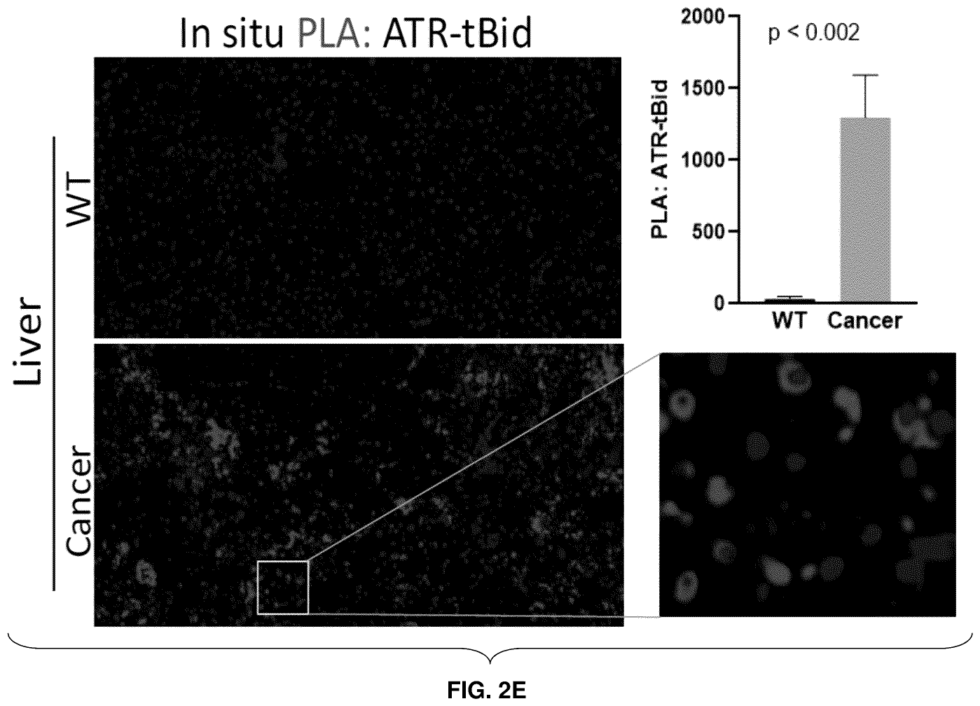

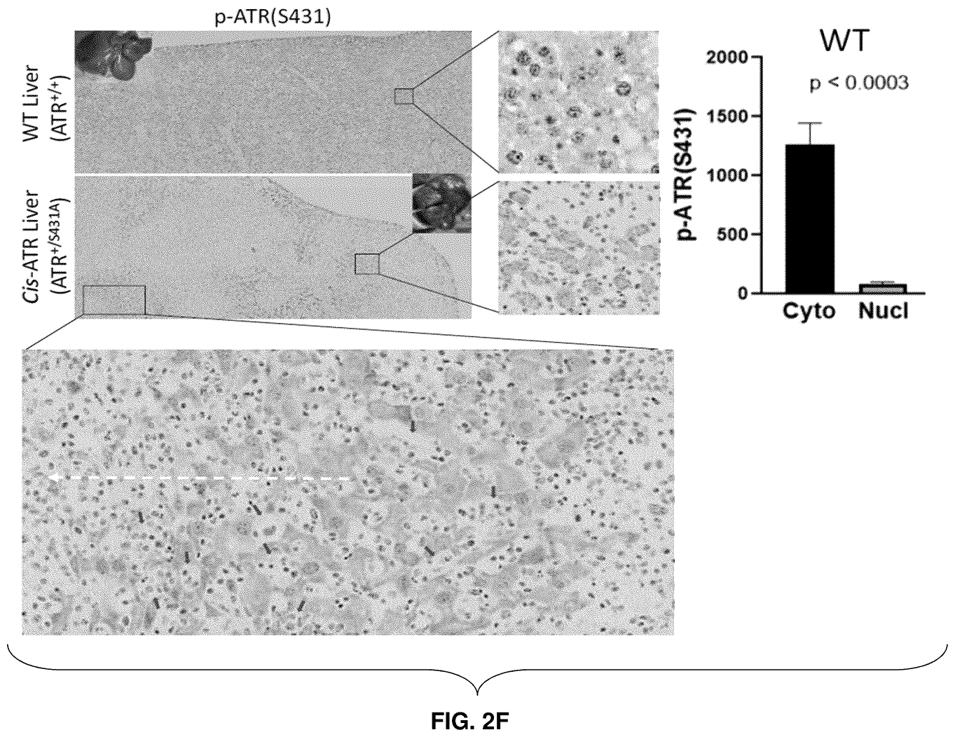

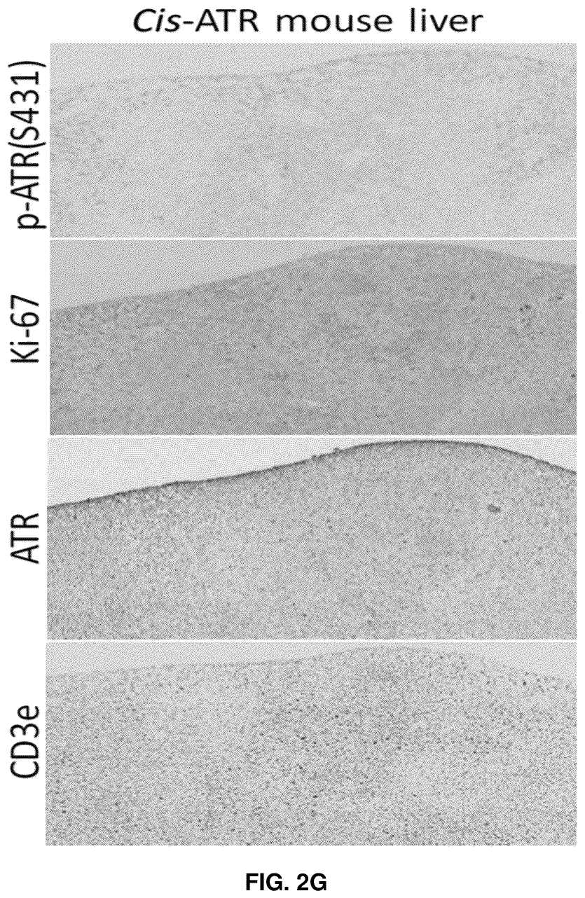

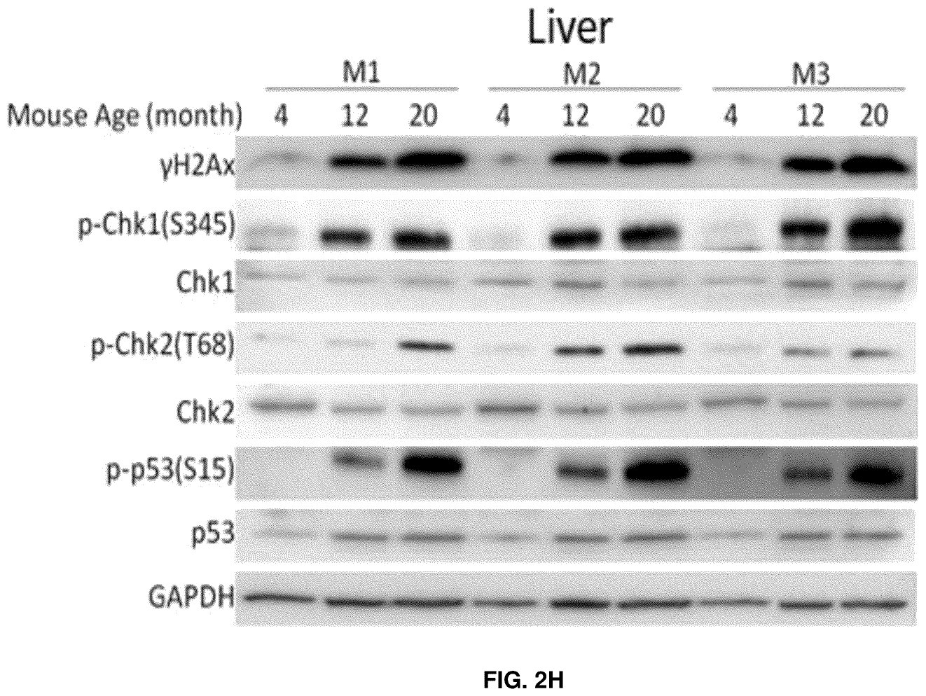

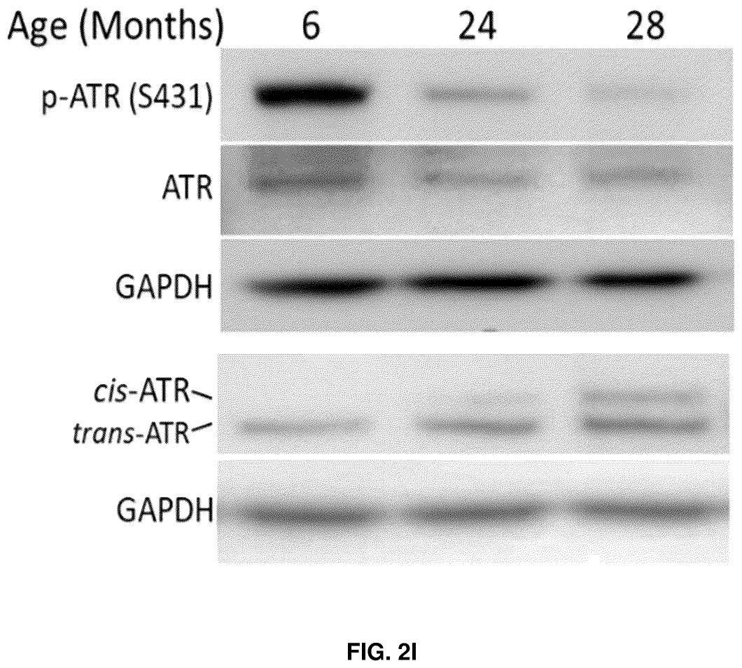

In contrast to trans-ATR mice, cis-ATR mice crossbred in a normal Mendelian distribution and grew healthily without noted abnormality to middle age. To examine whether older mice could develop abnormalities, cis-ATR mice were grown up to 26 months in two groups together with control wild-type (WT) mice: a 10-12-month age group and a 13-26-month age group. Strikingly, spontaneous tumors occurred in cis-ATR mice, particularly older mice. The most common tumor types were lymphoma and liver cancer. The left photo of FIG. 2A shows a representative enlarged, inflamed mesenteric lymph node tumor (attached to the intestinal mesentery). Tissue H&E staining shows a normal splenic architecture in WT mice, but not in the cis-ATR spleen and lymphoma, with the latter showing pleomorphic cells, nuclear atypia (FIG. 2A, middle panels, yellow arrows) and multinucleated giant cells (red arrows). Furthermore, IHC analysis shows higher levels of CD5, CD3e, and Ki-67 in lymph node tumor tissues versus WT spleen. There is no difference in staining with anti-CD20 antibody, indicating that the tumors are T-cell lymphoma or have a T-cell predominance. To confirm the clonality of the lymphoma, V(D)J rearrangements in the T-cell receptor (TCR) and B-cell receptor (BCR) genes were analyzed. Gel electrophoretic analysis of PCR-amplified DNA fragments shows differential amplification levels of rearranged TCR genes between WT mouse spleen (C) and lymphoma (L) of cis-ATR mice or between WT blood (B) and the blood from lymphoma-carrying (L) cis-ATR mice (FIG. 2B). In contrast, there were no noticeable differential BCR rearrangements.

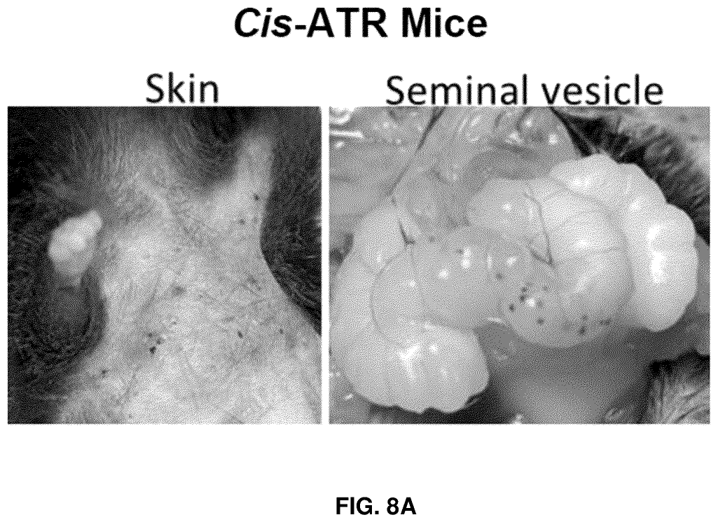

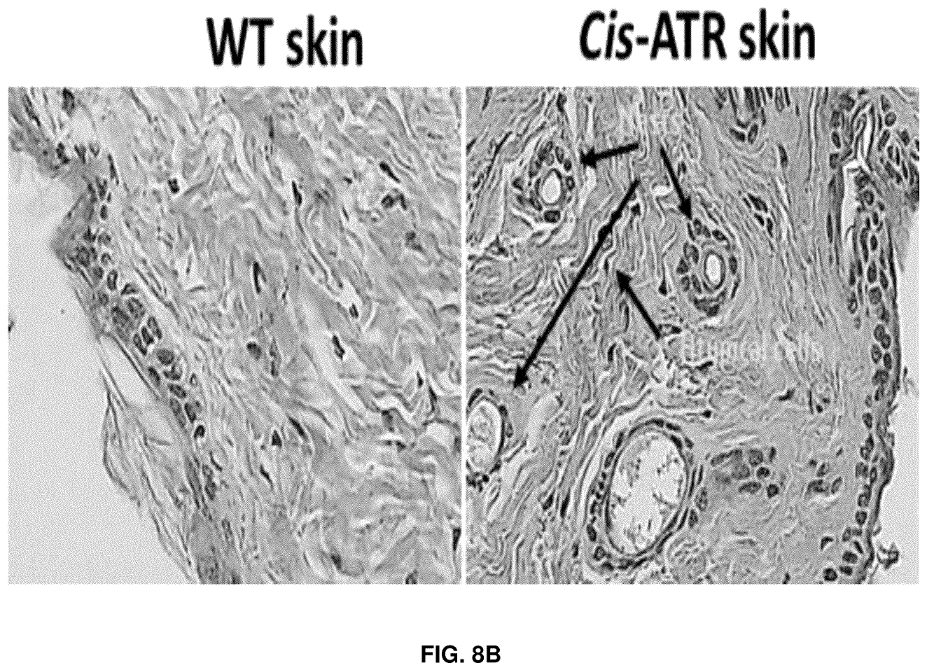

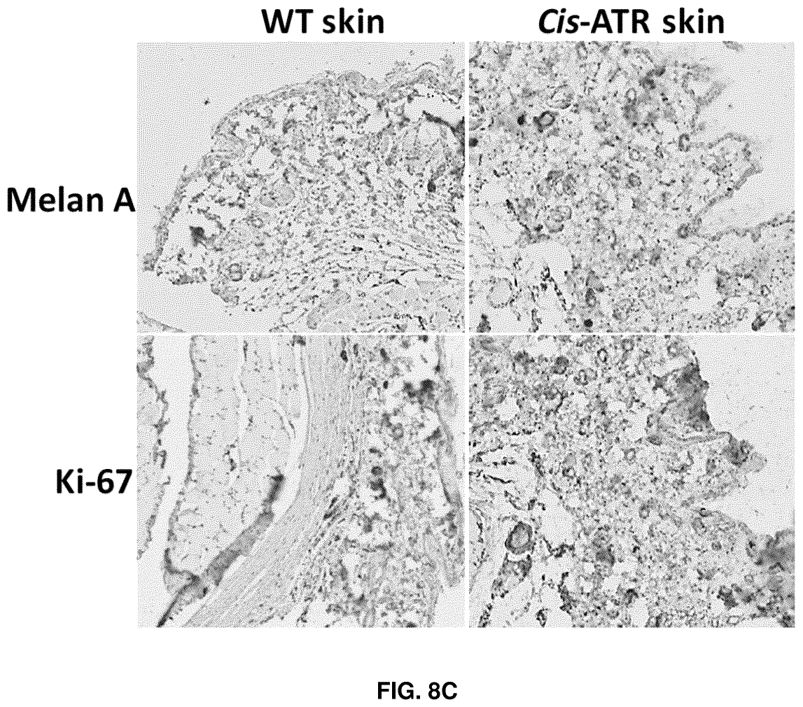

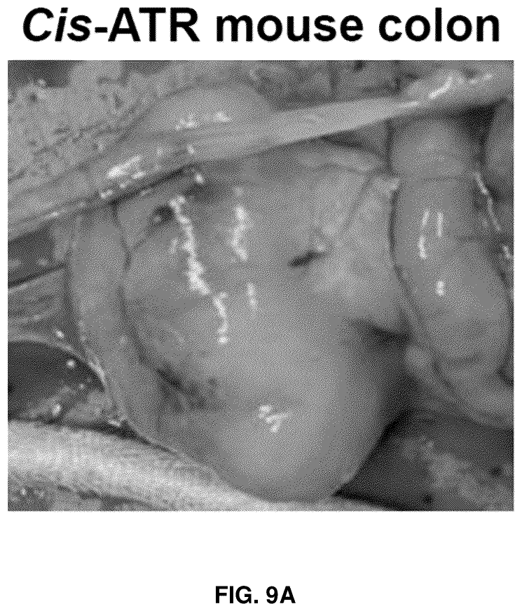

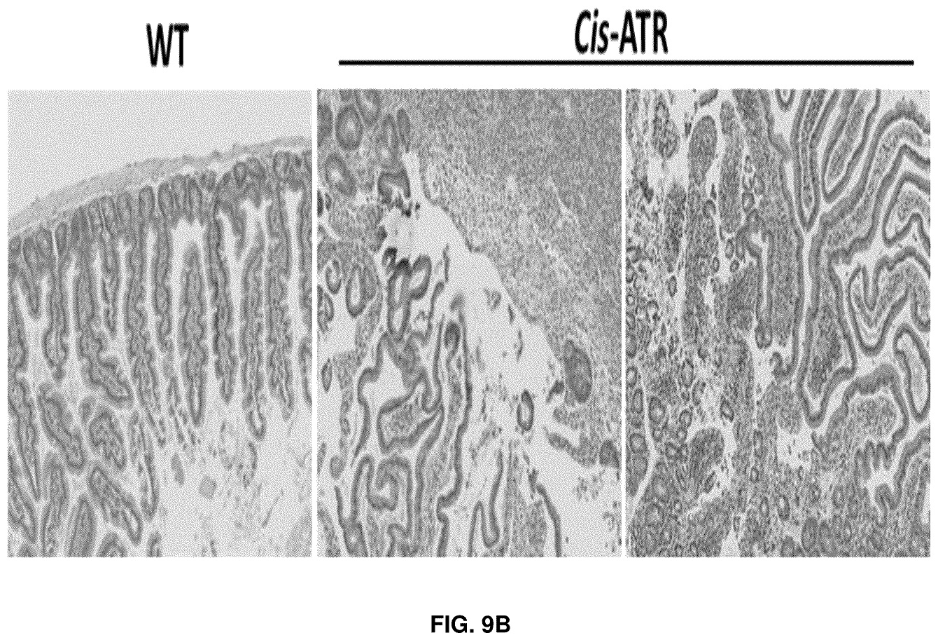

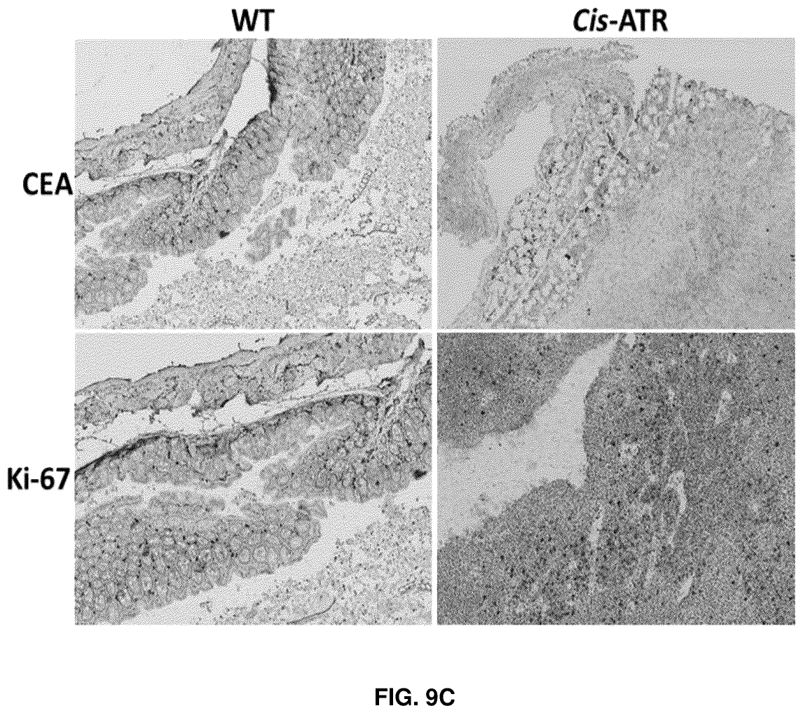

FIG. 2C shows two representative livers with tumors from cis-ATR mice. The enlarged livers exhibit surface color variegation and distinct tumor nodules. H&E staining shows a lymphocytic infiltration near a central vein (yellow arrow), pleomorphic cells, nuclear atypia, multiple giant cells, and mitotic figures (red arrows). Consistently, IHC analysis shows that tumor tissues stain positively for alpha-feto protein (AFP), a liver tumor marker, and CD3e, but not CD20. CD3e staining indicates infiltration of T-cells. Melanoma and colon tumors also grew in cis-ATR mice and were confirmed by cell and tissue morphology and melanoma marker melan A, the colorectal cancer marker carcinoembryonic antigen (CEA), and proliferation marker Ki-67 (FIGS. 8-9).

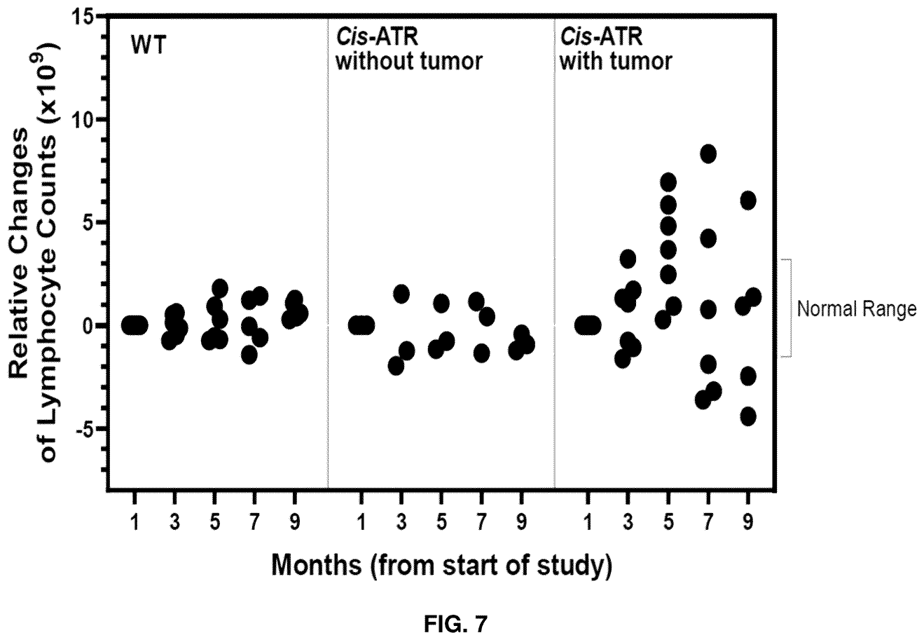

FIG. 2D summarizes the spontaneous cancer incidence of mice with specific genotypes. The data indicate that both WT and ATRP432A/+ mice are cancer free. In contrast, ATR^{S431A/+} or ATR^{S431A/S431A} mice are cancer prone. Importantly, cancer incidence increased dramatically with age, occurring in nearly 90% of older mice, but only in 18% of younger mice. Nevertheless, all mice were healthy into middle age with normal behaviors, appetite, weight, and blood lymphocyte counts. Remarkably, lymphocyte counts remain normal with the increasing age in the WT and cis-ATR mice without tumors, while the counts became significantly abnormal with increasing age for the cis-ATR mice with later-identified tumors (FIG. 7). To confirm that cis-ATR oncogenesis stems from cis-ATR's antiapoptotic activity, proximity ligation assays (PLA) were performed on WT liver and cis-ATR liver tumors to detect ATR-tBid interaction since cis-ATR antiapoptotic activity results from cis-ATR-tBid interaction at mitochondria. Significant ATR-tBid-induced PLA foci occurred in cancerous livers relative to WT livers (FIG. 2E). Remarkably consistent is the predominately-cytoplasmic location of the PLA foci, strongly indicating the correlation between spontaneous oncogenesis and cis-ATR-tBid interaction.

### Dephosphorylation of Cytoplasmic ATR(S431) as a Biomarker for Cancer Cells

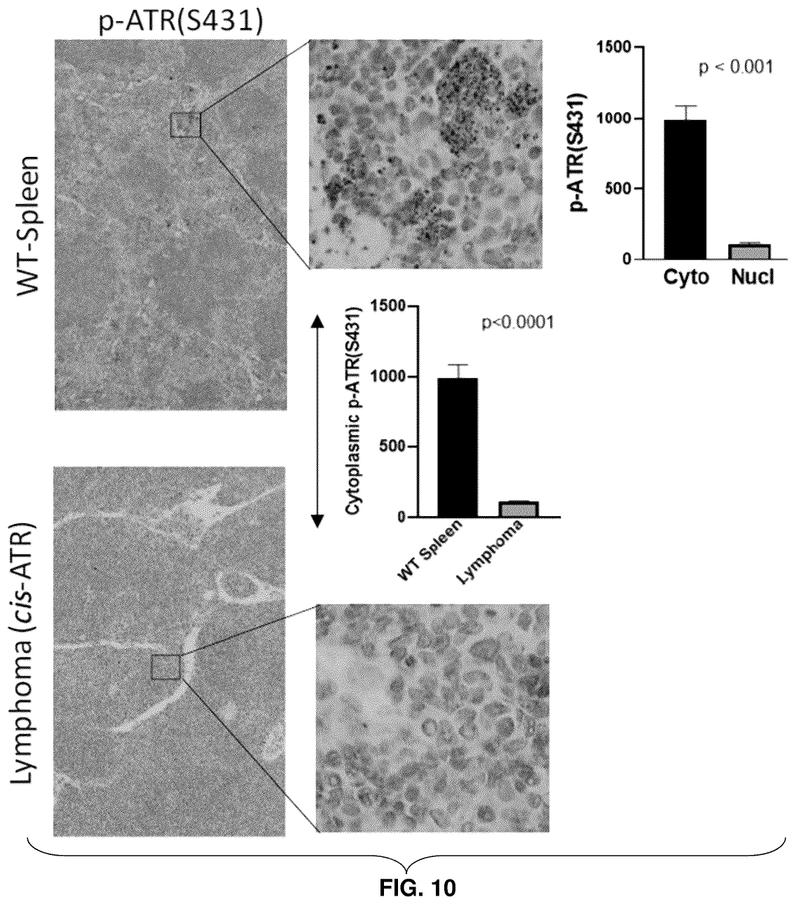

Dephosphorylation of human cytoplasmic ATR-S428 (p-ATR(S428)) increases cis-ATR formation as the dephosphorylation inhibits Pin1 conversion of cis-ATR to trans-ATR. IHC staining shows that the phosphorylation occurs homogeneously throughout the tissue in WT liver (FIG. 2F). Notably, p-ATR(S431) is present almost exclusively in the cytoplasm, indicating its importance in minimizing cytoplasmic cis-ATR via Pin1 isomerization (FIG. 5). In sharp contrast, a heterogeneous distribution of p-ATR(S431) occurs in liver cancer tissue from ATR^{+/S431A} mice, characterized by positive staining regions surrounding large unstained areas. Notably, staining intensity in these areas is significantly lower than that for WT tissue, likely due to the ATR^{+/S431A} heterozygosity (ATR^{+/S431A} mouse cells contain only ˜50% of wild-type cytoplasmic ATR that is phosphorable at S431). The unstainable areas, reflecting dephosphorylated ATR-S431, indicate cis-ATR dominance in the cytoplasm, protecting pre-oncogenic/oncogenic cells from apoptosis. This is confirmed by the IHC staining of cis-ATR mouse liver with Ki-67 antibodies (FIG. 2G), showing more cell proliferation in regions with low p-ATR(S431) in ATR^{+/S431A} mice in contrast to the homogeneous staining of total ATR. Interestingly, the light CD3e staining is primarily in the p-ATR(S431) staining areas, indicating a moderate infiltration of T-cell or T-cell lymphoma cells in the early oncogenesis regions. Consistently, close examination of the ATR^{+/S431A} IHC staining indicates that almost all the nuclei in these unstainable areas have aberrant morphology and are atypical or hyperchromatic (FIG. 2F, enlarged images). Even in the stainable areas, the normal-appearing nuclei with cytoplasmic p-ATR(S431) are surrounded by cells with abnormal and hyperchromatic nuclei (FIG. 2F). Remarkably, the enlarged IHC staining image illustrates the shift from normal to abnormal cell features. The intermediate cells displayed coarse heterochromatin aggregates (red arrows), which frequently occur in tumor cells. These intermediate cells feature enlarged nuclei and positive staining of p-ATR(S431) in the smaller, deformed cytoplasm. Such cells eventually lose cytoplasmic p-ATR(S431) staining (white dashed arrow), likely during the oncogenic process. The lack of cytoplasmic p-ATR(S431) staining also occurs in lymphoma tissue of cis-ATR mice (FIG. 10), indicating that dephosphorylation of cytoplasmic ATR-S431 may serve as a tumor biomarker.

### Role of Cis-ATR in Aging-Dependent Oncogenesis

The spontaneous oncogenesis in cis-ATR mice during aging due to cis-ATR inhibition of apoptosis, a critical physiological process for eliminating unstable cells, is relevant to normal aging-derived oncogenesis. Indeed, DNA damage and checkpoint signaling increase significantly during aging of WT mice (FIG. 2H). Accumulated DNA damage eventually may lead to potentially pre-oncogenic mutations in unstable cells which could be efficiently eliminated in normal tissues or organisms through apoptosis. It has previously been shown that DNA damage induces dephosphorylation of human cytoplasmic ATR-Ser428, correlating with cis-ATR formation. Consistently, the aging-dependent DNA damage does cause loss of p-ATR(S431) and cis-ATR accumulation in aging WT mice (FIG. 2I). The results in these examples show the dependence of spontaneous oncogenesis on cytoplasmic cis-ATR via aging-dependent DNA damage accumulation and ATR dephosphorylation, although the process is much slower in WT organisms.

### Antiapoptotic Function of Cis-ATR in Human

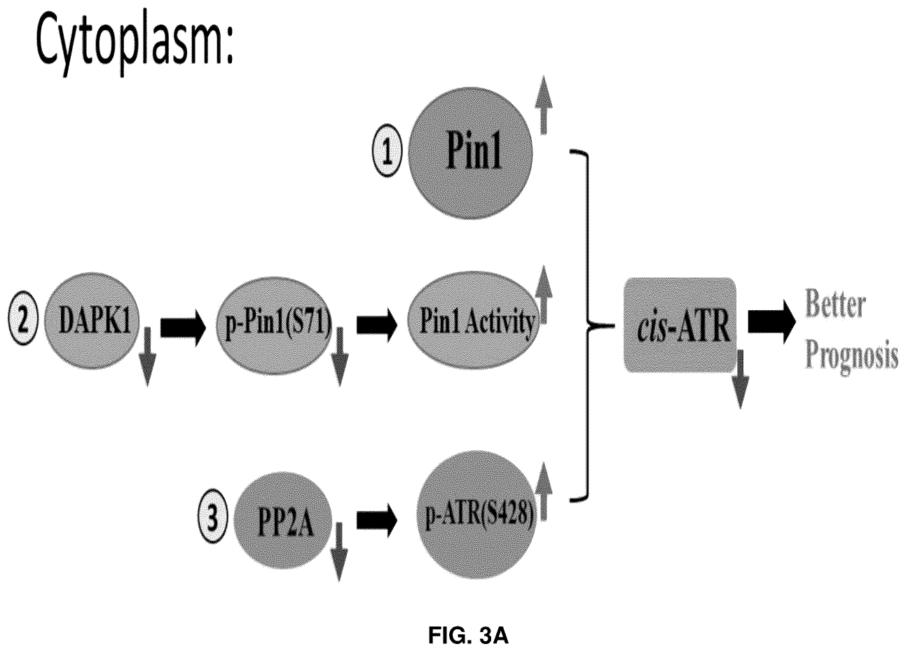

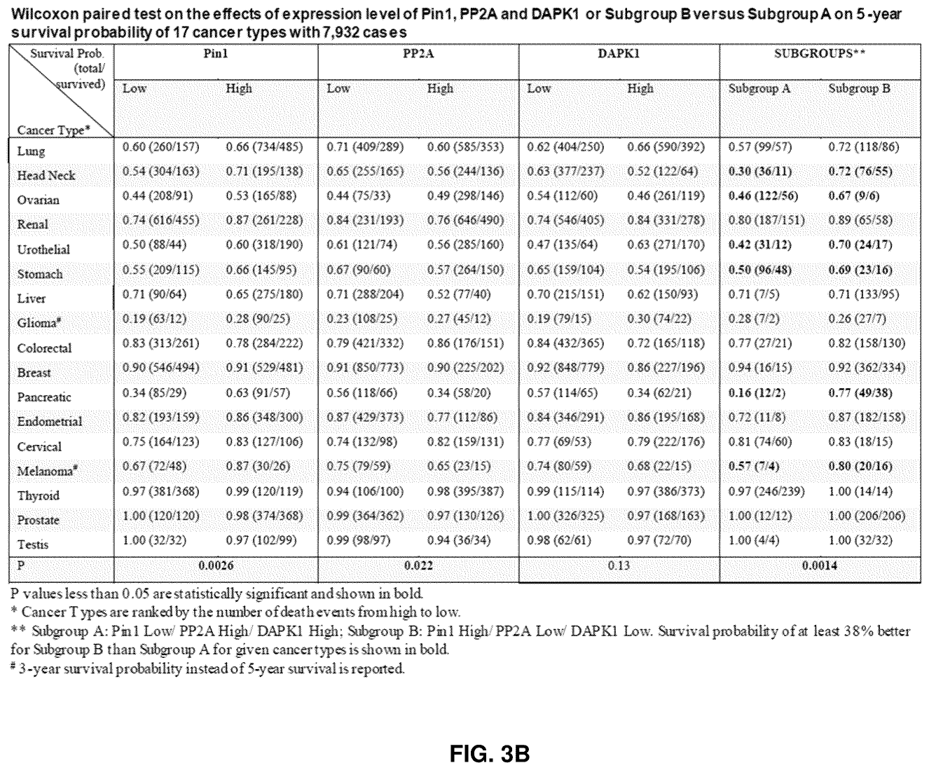

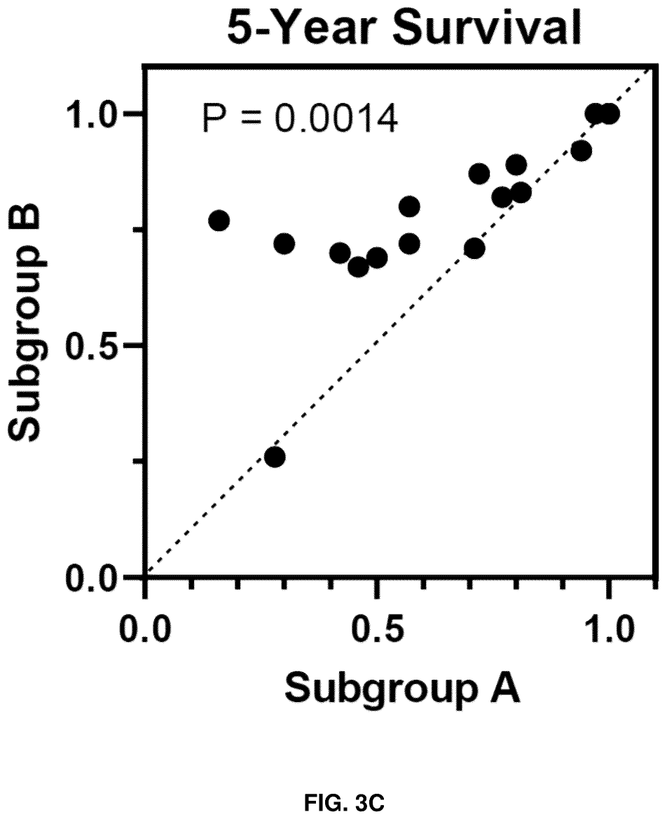

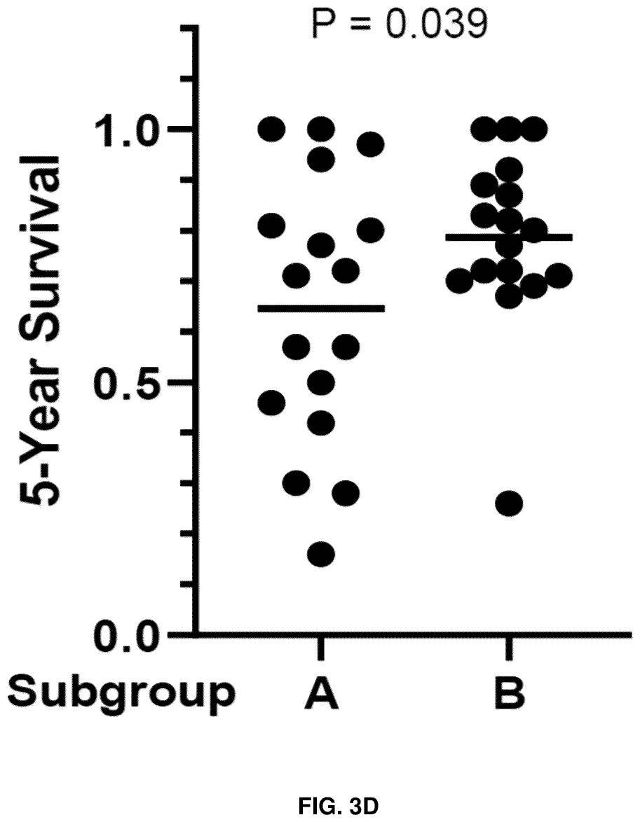

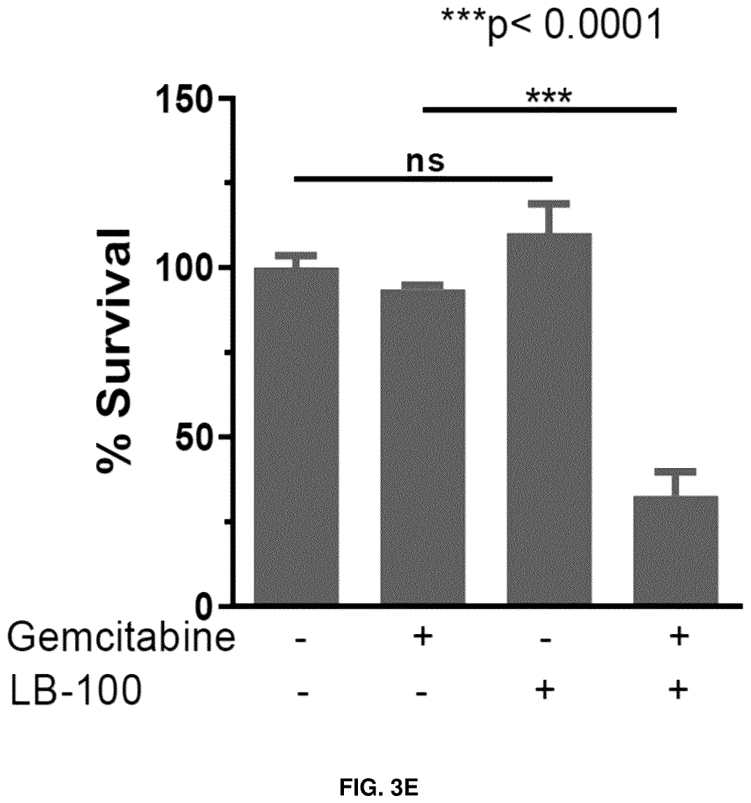

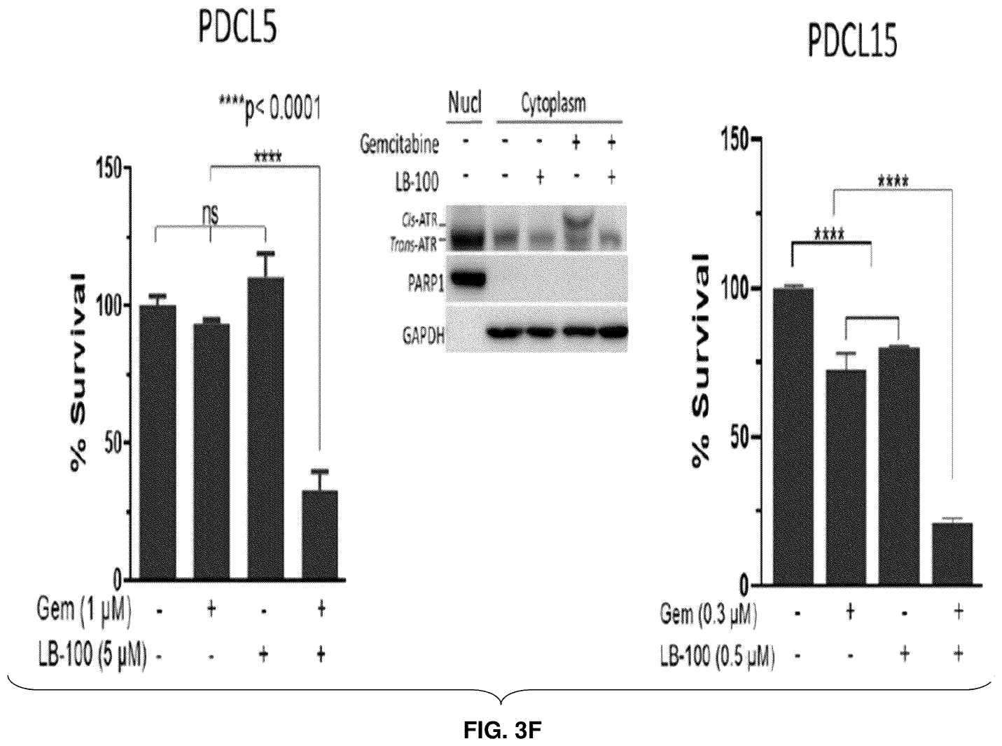

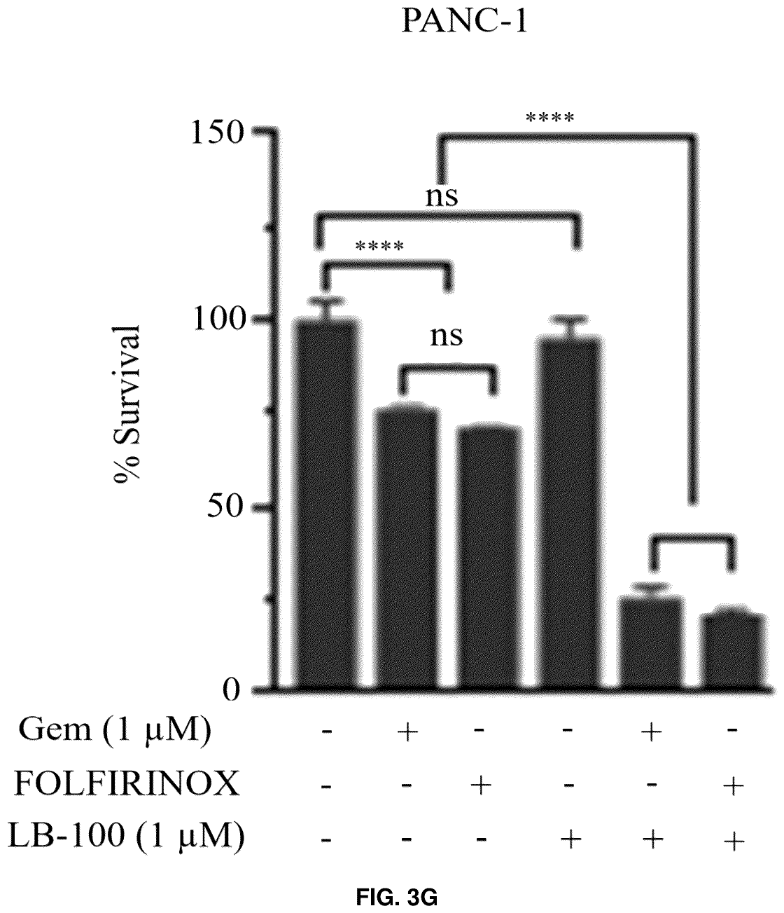

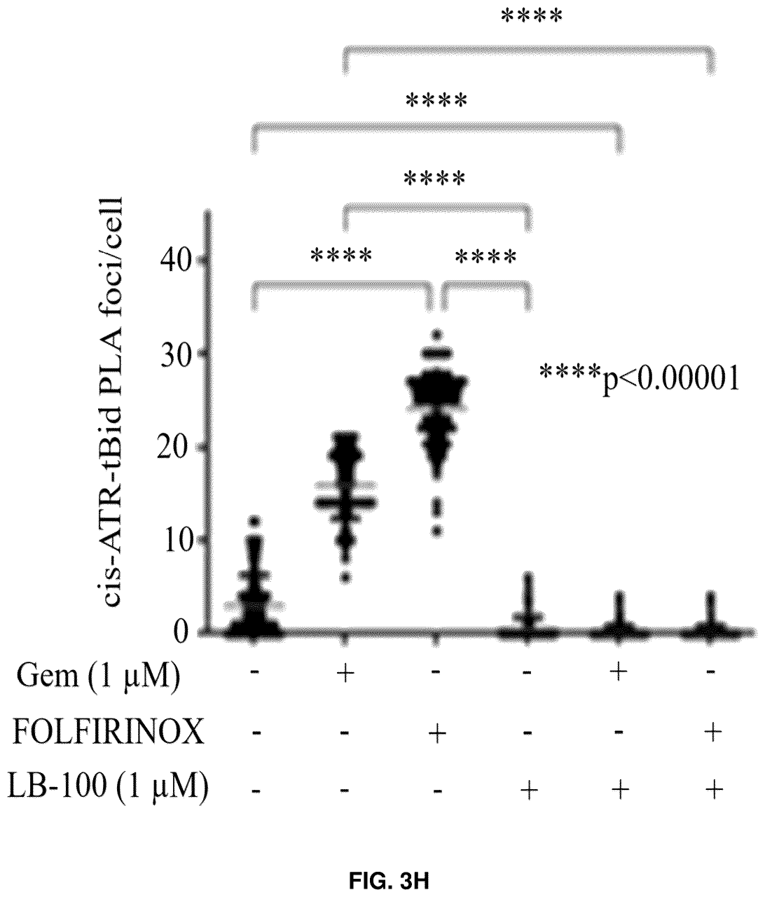

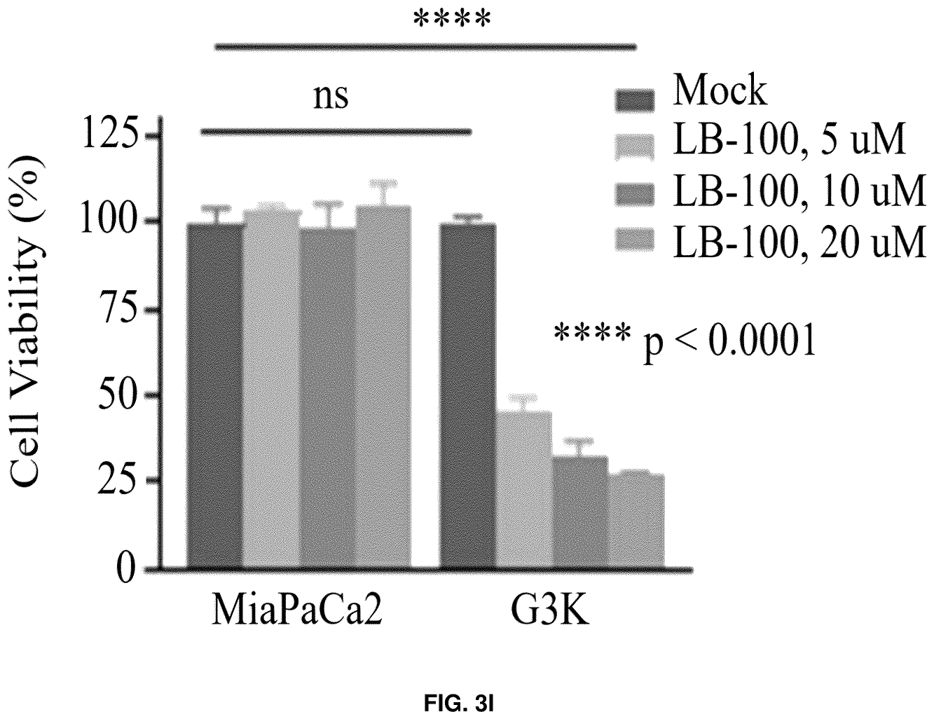

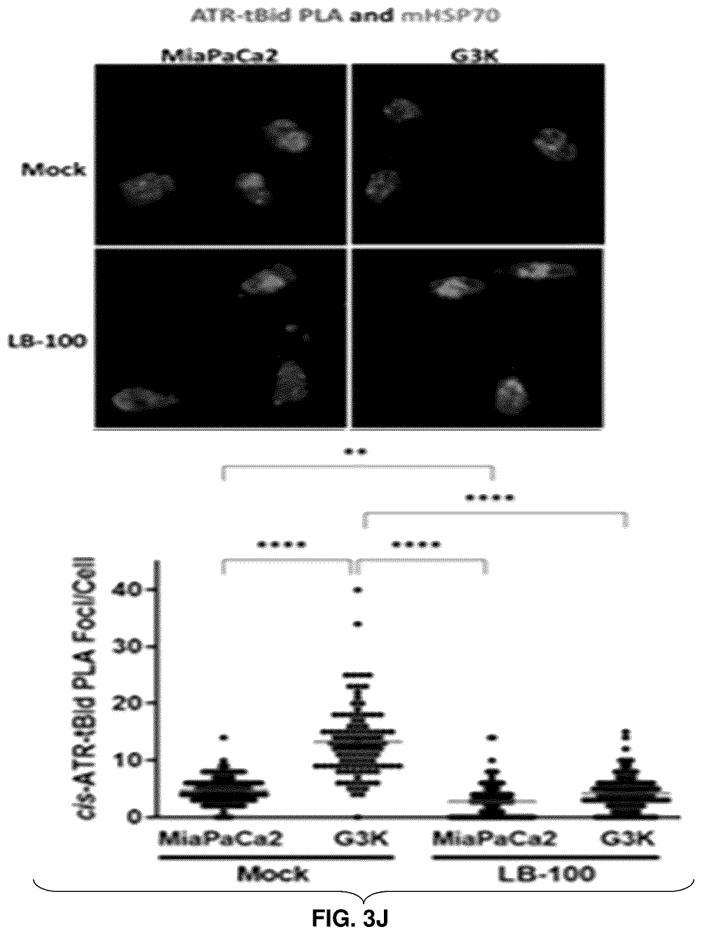

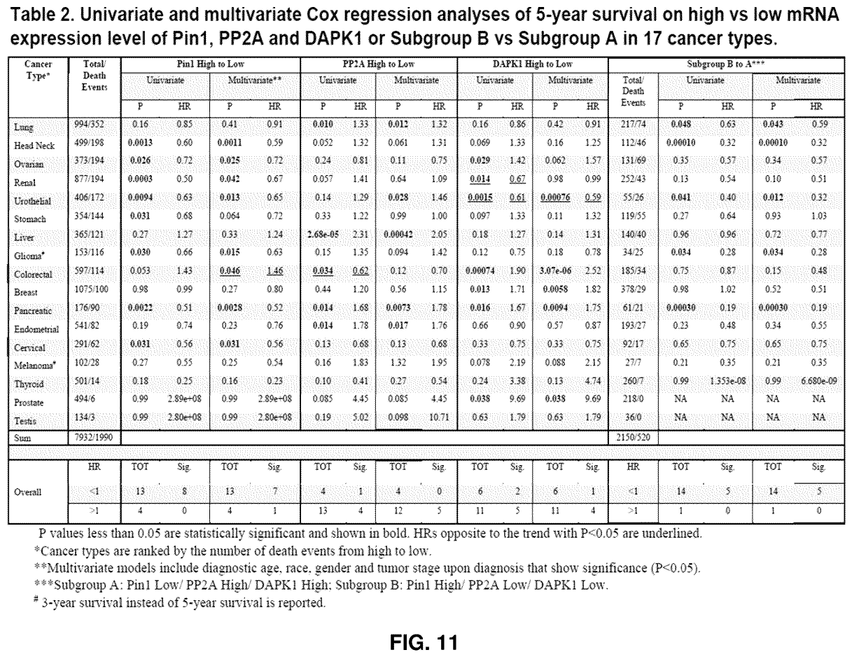

The above observations in mice raised the question of whether cis-ATR also is oncogenic in humans. As a newly identified protein, there is no human cancer data on cis-ATR levels currently available. However, cis-ATR is downregulated by Pin1, but upregulated by PP2A and DAPK1 (FIG. 5 and FIG. 3A). Since cis-ATR is antiapoptotic and pro-oncogenic, Pin1 is anti-oncogenic, while PP2A and DAPK1 are pro-oncogenic. To assess these correlations, the mRNA level of these three genes was assessed, in addition to patients' survival in 17 cancer types accounting for 7,932 total patients and 1990 death events in The Cancer Genome Atlas (TCGA) database (FIG. 6). In 13 of 17 cancer types, Pin1 high expression correlates to better survival as indicated by lowered hazard ratios (HR) in high Pin1 expression subgroups compared with low subgroups in both uncorrected univariate and corrected multivariate models against confounding factors including age, race, gender, and tumor stage upon diagnosis that show significance (P<0.05). Among these 13 cancer types, 8 and 7 types are statistically significant (P<0.05) in univariate models and multivariate models, respectively. In contrast, only 4 cancer types have increased HR in these subgroups with none significant in univariate models, and only 1 significant in multivariate models (FIG. 11, bottom).

PP2A has an opposite trend of Pin1. In 13 of 17 cancer types, PP2A high expression subgroups have increased HR and worsened survival compared with the low expression subgroups with 4 cancer types showing significance in univariate model. In the multivariate model, 12 cancer types show increased HR in high expression subgroups and 5 cancer types remain significant after correction (FIG. 11, bottom). Like PP2A, DAPK1 high expression is much more likely associated with worsened survival.

The above results indicate that Pin 1 high expression or PP2A or DAPK1 low expression favors better survival and supports the antiapoptotic role of cis-ATR in human subjects. To test whether there are synergistic effects among the 3 genes, whether the subgroups with simultaneously high Pin1 and low PP2A and DAPK1 expression (Subgroup B) gain advantage against the subgroups with simultaneously low Pin1 expression and high PP2A and DAPK1 expression (Subgroup A) was examined. In this analysis, the case number dropped to 2,150 with 540 death events in the two Subgroups. As a result, HRs can only be estimated in 15 of the 17 cancer types excluding prostate and testis cancer due to the lack of death events. In these 15 types, Subgroup B has better survival with lowered HR compared with Subgroups A with 5 cancer types showing significance, while only in 1 cancer type, Subgroup B, has increased HR but without significance in both univariate and multivariate models (FIG. 11, bottom). Despite smaller sample sizes, this outcome indicates that Subgroup B synergistically tends to do better than Subgroup A, and further supports the role of cis-ATR in oncogenesis.

To further validate the above observations are not at random, the 5-year survival probability (SP) of different subgroups was calculated for all 17 cancer types, and paired Wilcoxon rank-sum tests were performed. As shown in FIG. 3B, high Pin1 expression subgroups have significantly higher SP than low subgroups (P=0.0026), while PP2A high expression subgroups show significantly lower SP than low subgroups (P=0.022). The SP difference of DAPK1 expression level is not significant, likely because DAPK indirectly regulates cis-ATR while ATR is a direct substrate of Pin1 and PP2A. However, when analysis is conducted between the subgroups, patients in Subgroup B show significantly higher SP than Subgroup A with the smallest P value (P=0.0014) (FIGS. 3B, 3C). This remains true even for the analysis regardless of cancer types (FIG. 3D). These results indicate an accumulative synergy of these three genes and further support the antiapoptotic role of cis-ATR in oncogenesis. As a test of the analyses, patient derived PDCL5 cells of metastatic pancreatic cancer, which is largely affected by ATR isomerization (FIGS. 3B, 3C), were treated with the first line PDAC therapy drug gemcitabine, FDA-approved PP2A inhibitor LB-100, or the combination. Gemcitabine or LB-100 alone induces little cell death (FIG. 3E). Strikingly, however, their combination synergistically kills ˜70% cells, supporting the role of cis-ATR in cancer cell resistance.

FIGS. 3F-3J show a direct correlation between the drug sensitization and depletion of cis-ATR or cis-ATR-tBid complex levels in human pancreatic cancer cells. In addition, FIGS. 3I, 3J show that the drug resistant pancreatic cancer cells (G3K) acquired from gemcitabine treatment are extremely sensitive to cellular loss of cis-ATR for killing even in the absence of gemcitabine. FIGS. 3F-3J further support that cis-ATR plays an important role in drug resistance of cancer cells treated with DNA-damaging anticancer drugs and, thus, reducing the cellular (or specifically the cytoplasmic) levels of cis-ATR may overcome the drug resistances in cancer treatments.

### Differential Apoptotic Responses in Human Cis-ATR and Trans-ATR Cancer Cells

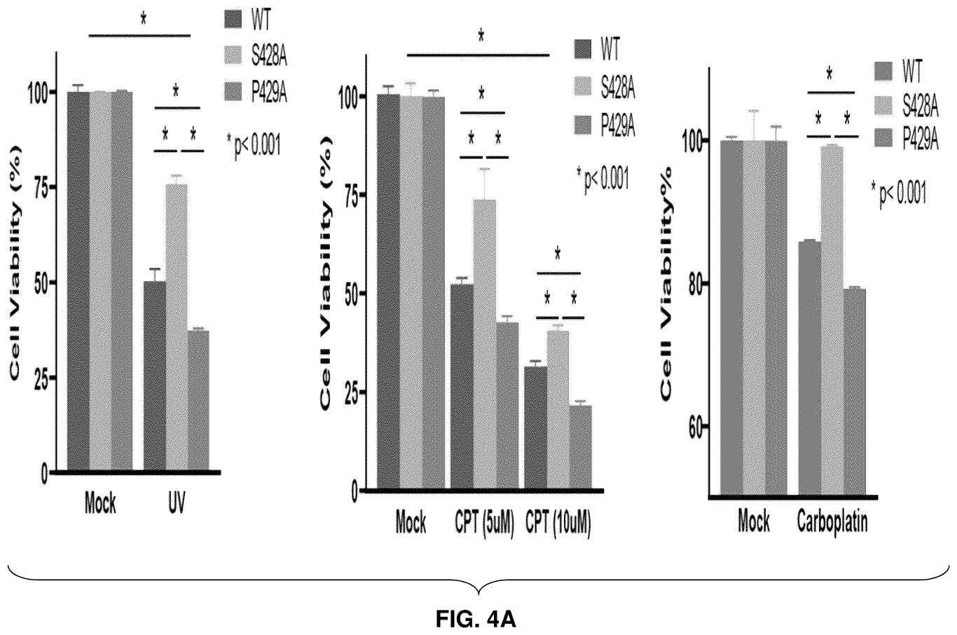

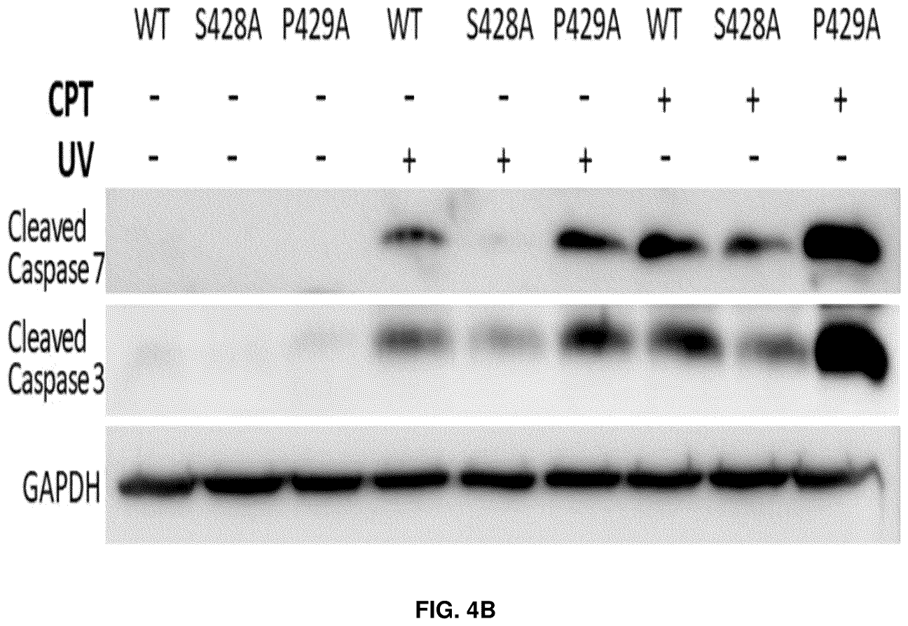

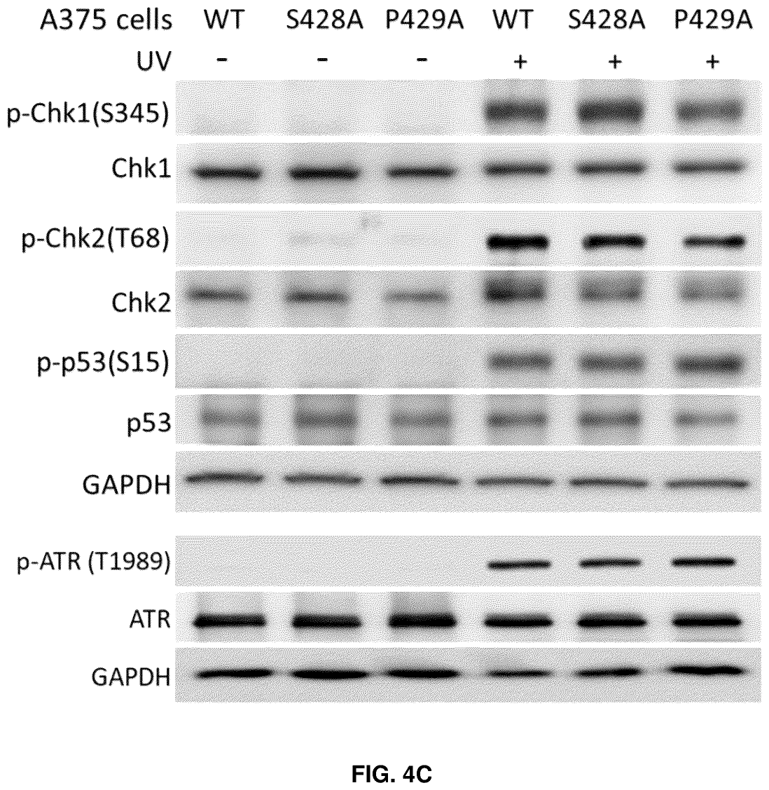

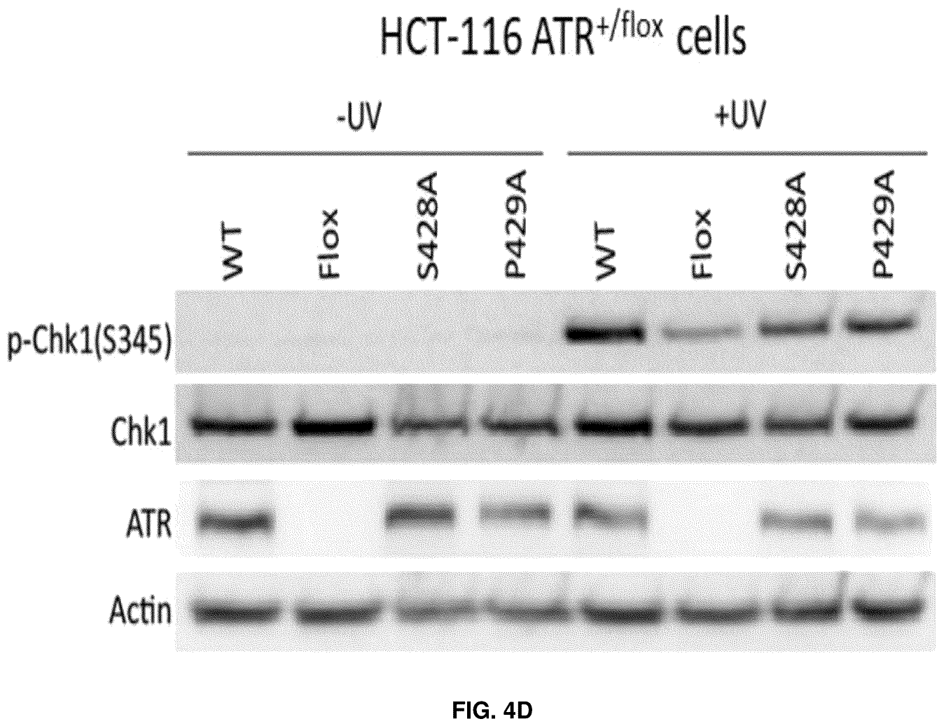

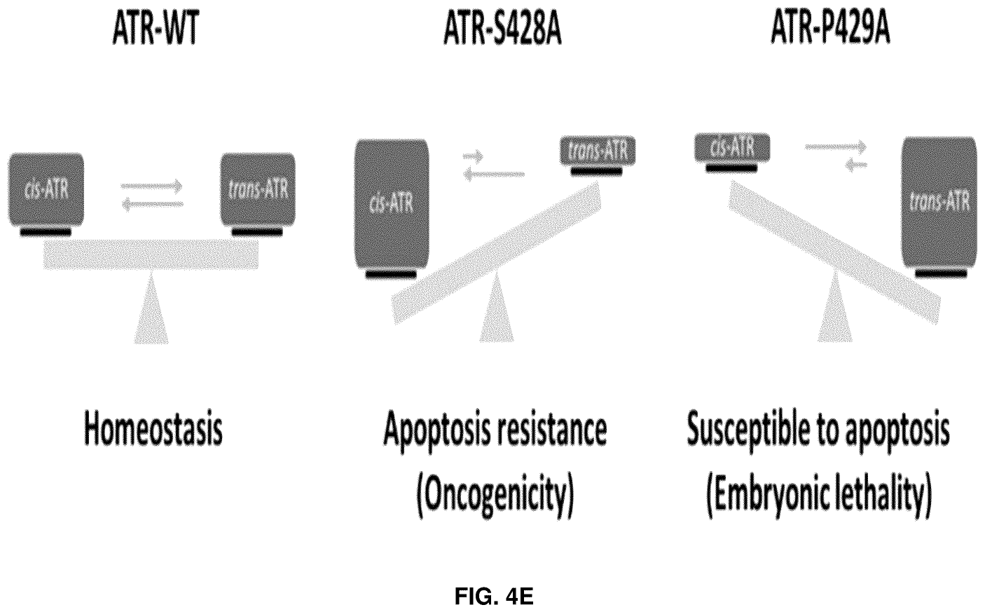

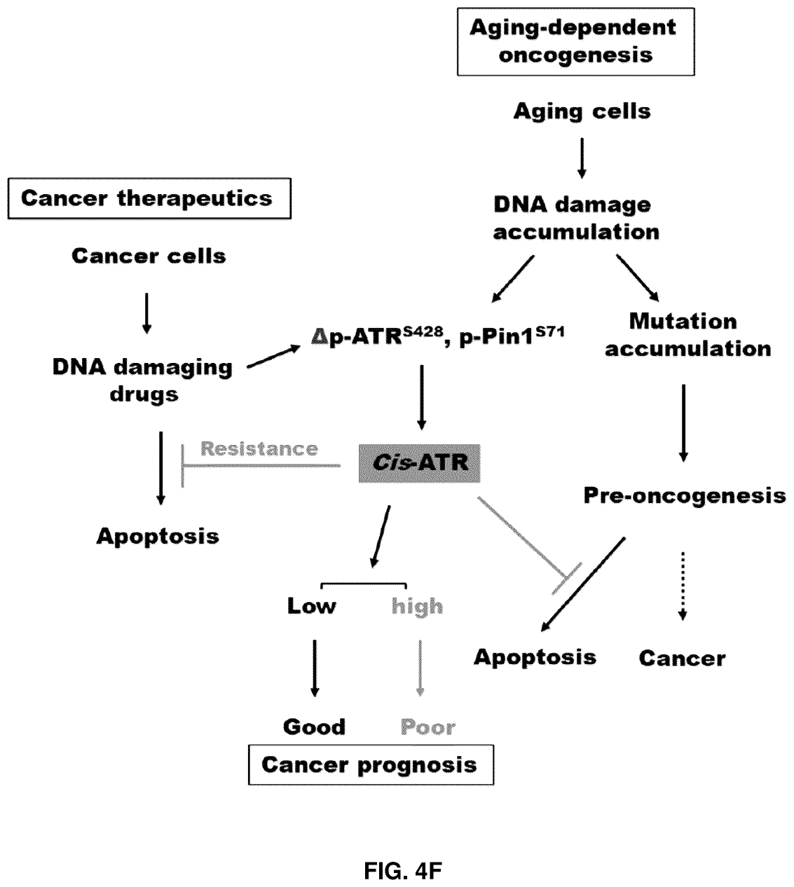

To confirm the significance of cis-ATR in human cancer, transgenic homozygous knock-in cis-ATR and trans-ATR human melanoma cells (A375) were generated. As shown in FIG. 4A, trans-ATR cells were significantly more sensitive to UV irradiation, camptothecin (CPT), and carboplatin, three DNA damage agents or therapeutic drugs, than WT cells; however, cis-ATR cells were the most resistant. These results are supported by the caspases 3&7 cleavages (FIG. 4B). Together, the data confirm cis-ATR as an antiapoptotic protein promoting cell resistance to DNA damage-induced apoptosis while trans-ATR does the opposite.

### Cis- and Trans-ATR Cells Show Intact DDR Checkpoint Signaling

The complete loss of ATR or its kinase activity leads to cell death through increased replication stress and a lack of checkpoint signaling. Is the embryonic lethality of ATR^{P432/P432A} due to a loss of this checkpoint activity? In addition, given the critical and indispensable role of ATR in DDR, an important question is whether the cytoplasmic cis-ATR dominance in cis-ATR cells compromised nuclear ATR kinase activity for DDR. Here, cis-ATR and trans-ATR A375 melanoma cells were treated with DNA damaging agents, followed by analysis of ATR-dependent DDR checkpoint signaling proteins. Strikingly, both cis- and trans-ATR cells showed DDR signaling equivalent to WT A375 cells (FIG. 4C). Alternatively, human colon cancer HCT-116-ATR^{flox/-} cells were transfected with ATR^{S428A} (cis-ATR) and ATR^{P429A} (trans-ATR) expression constructs, respectively, and then UV irradiated. Similar results were obtained as those from A375 cells (FIG. 4D), indicating that cytoplasmic cis-ATR formation not only is independent of ATR kinase activity, but also has no effect on ATR kinase-dependent DDR signaling in the nucleus. Together these results indicate that the ATR^{P432A/P432A} embryonic lethality is independent of ATR kinase activity status and different from the embryonic lethality caused by the loss of ATR kinase activity.

### Discussion

These examples reveal a mechanism demonstrating the importance for cells to precisely balance cis-ATR and trans-ATR, two natural ATR isomers, for cellular homeostasis and organism wellbeing. An imbalance may increase the risk of diseases associated with cell death and immortality (FIG. 4E). These examples also highlight that as the last defense against oncogenesis, cells may need a base level of cytosolic cis-ATR only sufficient to prevent death due to normal genomic stresses. Otherwise, abnormally high cis-ATR could significantly predispose cells to cancer during aging or genotoxic insults, while loss of cis-ATR could promote cell death-related diseases. The normal DDR checkpoint signaling found in cis-ATR and trans-ATR cells indicates that oncogenesis may occur even with intact DDR checkpoints. Furthermore, cis-ATR level may increase during natural aging of cells or upon cancer therapeutics with DNA damaging agents. The results show that cis-ATR is a target for cancer therapeutics as its antiapoptotic activity can protect pre-cancerous and cancer cells from killing.

While trans-ATR^{P432A}, due to a single residue substitution, is chemically different from trans-ATR^{+/+}, both are sterically and functionally identical (FIGS. 1D, 2E, 2I, 4C, 4D). The same is true for cis-ATR^{+/+} and cis-ATR^{S431A}. Since trans- and cis-ATR are interconvertible in WT cells, gain of trans-ATR means loss of cis-ATR, and vice versa. Therefore, the ATR^{P432A/P432A} embryonic lethality is likely not caused by the presence of trans-ATR which is the nuclear ATR kinase, but instead by the absence of antiapoptotic cis-ATR (a cis-ATR^{null}) in the all trans-ATR containing embryos (FIGS. 4A, 4B). This lethality is mechanistically different from the embryonic lethality of ATR^{-/-} mice lacking both trans-ATR and cis-ATR, and from the kinase-dead ATR^{+/KD} mouse infertility making ATR^{KD/KD} mice nonexistent. Unlike trans-ATR cells, a characteristic of cis-ATR cells is the dominance of cis-ATR in the cytoplasm but not in the nucleus where trans-ATR is always the only ATR form. In fact, the trans-ATR embryonic lethality serves as a perfect control to confirm the critical antiapoptotic role of cis-ATR in protecting cells from death.

The oncogenesis phenotypes demonstrated by the aging cis-ATR mice highlight the role of cis-ATR in aging-dependent oncogenesis. As illustrated in FIG. 4F, this role is defined by mechanisms including 1) age-dependent increase in DNA damage and accumulation of DNA mutations, and 2) DNA damage increases cytoplasmic cis-ATR via dephosphorylation of p-ATR^{S428} and inhibitory phosphorylation of Pin1^{S71} (FIGS. 2E-2I, 10). Inhibitory Pin1^{S71} phosphorylation increases with age. The DNA damage-induced cis-ATR upregulation disrupts the homeostatic trans- and cis-ATR balance, and blocks apoptosis, allowing cells with silent pre-oncogenic mutations and genomic instability to bypass death and become oncogenic in either normal or cis-ATR mice. The difference is that WT mice accumulate less cis-ATR so that DNA damage may trigger apoptosis against basal cis-ATR to eliminate the potentially oncogenic cells, thus lowering cancer incidence. However, the oncogenesis mechanisms are possibly the same for both types of mice. The tumor types observed here include lymphoma, colon, melanoma, and liver, as these organs have relatively high cell replication and regeneration rates and, thus, high chance of DNA mutagenesis during aging. In addition, cis-ATR or pATR-S428 dephosphorylation serves as a biomarker for cancer prognosis (FIG. 4F). Furthermore, since many cancer therapeutic drugs are DNA damaging agents, DNA damage-augmented cis-ATR formation may lead to drug resistance by suppressing apoptosis (FIG. 4F).

The mining on the TCGA data provides further support of the oncogenic role of ATR in human subjects. For the 17 investigated cancer types, high expression of Pin1, low expression of PP2A, or DAPK1 tend to favor patients' survival either respectively or synergistically, although more mRNA does not guarantee more protein in cells. The Wilcoxon paired test on the 5-year SP indicates this is true because more immediate factors regulating ATR isomerization (Pin1, PP2A) show significance while the indirect mediator DAPK1 does not (FIG. 3B). Advantageously, cis-/trans-ATR imbalance may contribute to deadly cancers such as glioma and pancreatic cancer. Intervention of ATR isomerization may provide avenues of successful treatments for these cancers.

Cis-ATR is an addictive oncogenic protein in cancer cells. After all, most, if not all, cancer cells depend on resistance to apoptosis to survive during cancer treatments. Cis-ATR's antiapoptotic activity at mitochondria occurs far downstream from DDR pathways. Since cis-ATR blocks apoptosis execution, oncogenesis due to either the genetic defects or spontaneous mutations in upstream pathways may depend on the antiapoptotic activity to silence apoptosis. This may also be true in cancer treatments as many cancer therapeutics target the signaling pathways upstream to apoptosis execution, implicating cis-ATR as a common endpoint target to sensitize cancer therapeutics. Since cis-ATR has no effect on ATR kinase-dependent DDR checkpoint activities, targeting of cis-ATR should have minimal adverse effects.

The present examples have also established a unique mouse model for aging-dependent oncogenesis. Cis-ATR^{S431A}, which mimics the native cis-isomeric form of ATR, may not interfere with upstream cellular pathways. Defects in these pathways may lead to cancer during aging. Blocking of apoptosis by cis-ATR to prevent pre-cancerous cell death allows the oncogenic potential to be expressed, providing an opportunity to study the mechanisms of how aging leads to oncogenesis amid DNA damage accumulation, and to identify, within a shortened time period, which pathways are intrinsically compromised towards oncogenesis during aging.

### Methods

### Generation of the ATR Knock-In Mice and Melanoma Cell Lines

ATR-S431A and ATR-P432A single amino acid substitution C57BL/6 mice were generated using CRISPR-Cas9 gene editing technology. The sgRNA was designed and the S431A and P432A mutant mice were generated via the Transgenic Animal and Genome Editing Facility Core at Cincinnati Children's Hospital Medical Center. Mice were in-bred through a series of generations to eliminate any mosaic genotypes. Restriction cleavage assays using AfeI or SfoI were employed to determine heterozygosity and homozygosity of S431A mice and heterozygosity of P432A mice. The results were confirmed by DNA sequencing. AfeI is P432A genotype specific and SfoI is S431A genotype specific. The genetic codons CCT in ATR-S431A and CCA in ATR-WT both code for proline, while the codons AGC in ATR-P432A and TCA in ATR-WT both code for serine.

The melanoma knock-in cell lines (ATR-S428A and ATR-P429A) were generated using CRISPR-Cas9 technology from the human A375 melanoma cell line. The sgRNA was validated and cell lines generated by the Genome Engineering and iPSC Center (GEiC) at the Washington University at St. Louis.

### Cell Culture, Drug and UV Treatments, Antibodies

All cell lines were cultured at 37° C., 5% CO₂. A549, A375 WT, and mutant cell lines were maintained in a base medium of DMEM, while HCT 116 WT and floxed cell lines were grown in a base medium of McCoy's 5a modified medium. To make the complete growth medium, fetal bovine serum to a final concentration of 10% and penicillin and streptomycin to 1% each were added to all base media. Camptothecin (CPT) (Sigma-Aldrich C9911) treatments were performed at 5 and 10 μM final concentrations for 16 hours. UV irradiation was performed using a 254 nm lamp at 40 J/m², followed by a 2-hour recovery (UV-induced cis-ATR formation assays) or 60 J/m² with a 24-hour recovery (apoptosis assays). Antibodies for immunoblotting were utilized as advised in their respective protocols; pATR (S428) (Cell Signaling Technology, 2853), p-ATR (T1989) (Cell Signaling Technology, 58014), ATR (Bethyl Laboratories, Inc., A300-137A and A300-138A), phospho-Chk1 (S345) (Cell Signaling Technology, 2348), Chk1 (Cell Signaling Technology, 2360) Phospho-Chk2 (Thr68) (Cell Signaling Technology, 2197), Chk2 (Cell Signaling Technology, 2662), p-p53 (S15) (Cell Signaling Technology, 9284), p53 (Cell Signaling Technology, 2524), phospho-Histone H2A.X (Ser139) (Cell Signaling Technology, 9718), GAPDH (Santa Cruz Biotechnology, sc-47724, PARP1 (Cell Signaling Technology, 9532), cleaved caspase-3 (Asp175) (Cell Signaling Technology, 9664), cleaved caspase-7 (Cell Signaling Technology, 9491), BID (C-20) (Santa Cruz Biotechnology, sc-6538), and β-actin (Invitrogen, MA1-140).

### Western Blotting (WB)

Cultured cells were harvested by trypsinzing or scraping into the appropriate buffers. Whole cell lysis was done with buffer containing: 50 mM Tris-HCl, pH 7.8, 150 mM NaCl, 1 mM EDTA, 1% Triton X-100, and protease/phosphatase inhibitor cocktail 1× (Thermo Fisher Scientific, 78430). Incubation was done at 4° C. for 30 minutes, then centrifuged at 10000 rpm for 10 min.

Whole cell proteins were extracted from mouse tissues in lysis buffer (as above) after tissue processing with an electric hand-held tissue homogenizer (Sigma-Aldrich, Z742486). Then, incubation for 1 h and centrifugation at 14000 rpm for 10 minutes, both at 4° C., were undertaken. To denature proteins, 2× or 4×SDS loading buffer was added to the lysates which were then boiled at 95° C. for 5 min. SDS-PAGE electrophoresis was carried out in 8 or 12% Tris-glycine SDS gels or 3-8% Tris-Acetate SDS PAGE gradient gels (Invitrogen, EA0378). Proteins were transferred to PVDF membranes (Amersham Hybond P 0.45 PVDF, 10-6000-29) and immunoblot analysis was performed with primary antibodies directed against several proteins. Chemiluminescence was done using SuperSignal West Pico Chemiluminescent Substrate (Thermo Fisher Scientific, 34580) and immunoblots were visualized with the GE Amersham Imager 680.

Cell counting was done with 0.4% trypan blue using chamber slides for the automated Countess™ Automated Cell Counter from Invitrogen (C10228). Complete blood cell counts from mice blood employed the Abaxis VetScan HM5C Hematology Analyzer following the manufacturer's protocols.

### Apoptosis Assays

MTT viability assays were performed according to the manufacturer's specifications (Cayman's MTT Proliferation Assay kit #10009365), and fluorescence was measured by a spectrofluorometer. At least three independent experiments were conducted, and cells were plated at least in triplicates per condition being tested. Statistical analysis of samples for standard deviations was performed with Student's t test, and a value of P<0.05 was considered significant.

### Duolink In Situ Proximity Ligation Assay

FFPE tissue sections were analyzed in the Duolink in-situ detection of protein-protein interactions, according to the manufacturer's instructions (Sigma-Aldrich, DU092008). Images were captured using the Olympus VS120 Slide Scanner Slide Analyzer.

### Cellular and Tissue Fractionation

For mouse tissue fractionations, fresh tissue samples cryopreserved at necropsy were used according to the instructions for the Subcellular Protein Fractionation kit for Tissues's (Thermo Fisher Scientific, 87790), but for cell line fractionation, differential centrifugation into cytoplasmic and nuclear isolates using different lysis buffers was done at 4° C.

To obtain the cytoplasmic fraction, a hypo-osmotic cytoplasmic lysis buffer (10 mM HEPES, pH 7.9, 10 mM KCl, 3 mM CaCl₂, 1.5 mM MgCl₂, 0.34 M sucrose, 10% glycerol, 0.1% Triton X-100) with 1× protease and phosphatase inhibitor cocktail (Thermo Fisher Sci) was added to packed cells, at a ratio of 10 volumes buffer:1 volume packed cell, and incubated for 10 min at 4° C. The suspension was centrifuged for 7 min at 600×g and the cytoplasm-containing supernatant collected. The pellet (nuclei fraction) was washed twice in ice-cold cytoplasmic lysis buffer, then lysed with 1/10 volume of the nuclear lysis buffer (50 mM Tris-HCl, pH 7.9, 140 mM NaCl, 3 mM CaCl₂). After rotation for 20 min at 4° C. the supernatant was collected after centrifugation at 10,000 rpm for 10 min at 4° C. 2×SDS loading buffer was added to both fractions, which were then boiled at 95° C. for 5 minutes.

To ascertain successful cellular fractionations, PARP1 and GAPDH were probed for, and GAPDH also was used to normalize equal protein loadings.

### Tissue Processing and ATR Genotyping

Mice ear punch tissue specimens were obtained and stored at 80° C. Other mice tissue and tumor specimens and blood were obtained at the time of necropsy, snap-frozen, and stored at −80° C. DNA was prepared from tissues using the DNeasy Blood and Tissue Kit (Qiagen, 69506). ATR genotyping was done using HaeII (New England BioLabs Inc., R0107) restriction enzyme digest on PCR product. The ATR primer pair used for the PCR amplification were forward-Atrgenf1 GACTCATGTAACACCTCATGCA (SEQ ID NO: 1) and reverse-Atrgenr2 ACCCAAATTAAACAGGCATGC (SEQ ID NO: 2) for a product size of 459 bp. Amplifications reactions were performed with Phusion® High-Fidelity DNA Polymerase (New England BioLabs Inc., M0530) in a Applied Biosystems thermal cycler (Thermo Fisher Scientific). The reaction volume was 20 μl with conditions: 98° C. for 5 min, followed by 40 cycles consisting of denaturation at 98° C. for 15 s, annealing at 62° C. for 15 s, extension at 72° C. for 30 s, and a final extension at 72° C. for 7 min. PCR products were electrophoresed in 2% agarose gels, using 0.5×TBE buffer and visualized by ethidium bromide staining. Following visualization, DNA bands were excised from the agarose gels for DNA purification as per Qiaquick Gel Extraction kit protocols (Qiagen, 28706) and eluted DNA was sequenced to confirm the ATR genotype. Some mice tissue and tumor samples also were collected at necropsy for formalin fixation, paraffin embedding and slide processing.

### Gene Rearrangement Analysis

Genomic DNA from mice blood, spleen, and tumor tissues were isolated using DNeasy Blood and Tissue Kit (Qiagen) according to manufacturer instructions. Differences in TCR rearrangements among various samples were analyzed using 50 ng of genomic DNA as template. PCRs were performed in a volume of 50 μl for 35 cycles (30 seconds 98° C., 30 seconds at 55° C., and 1 minute at 72° C.) using the following primer pairs 25: Vγ1.1: 5′-GAGAGTGCGCAAATATCCTGTATA-3′ (SEQ ID NO: 3) and Jγ4: 5′-TGGGGGAATTACTACGAGCT-3′ (SEQ ID NO: 4); Vγ2/4: 5′-TATGTCCTTGCAACCCCTAC-3′ (SEQ ID NO: 5) and Jγ1: 5′-ATGAGCTTAGTTCCTTCTGC-3′ (SEQ ID NO: 6); Vγ1.2: 5′-GTGCAAATATCCTGTATAGTT-3′ (SEQ ID NO: 7) and Jγ2: 5′-ACAGTAGTAGGTGGCTTCAC-3′ (SEQ ID NO: 8); Vγ5/7: 5′-ATGAAGGCCCGGACA-3′ (SEQ ID NO: 9) and Jγ1: 5′-ATGAGCTTAGTTCCTTCTGC-3′ (SEQ ID NO: 6); Vγ4/6: 5′-ACAAGTGTTCAGAAGCCCGA-3′ (SEQ ID NO: 10) and Jγ1: 5′-ATGAGCTTAGTTCCTTCTGC-3′ (SEQ ID NO: 6); Vγ3/5: 5′-TGGATATCTCAGGATCAGCT-3′ (SEQ ID NO: 11) and Jγ1: 5′-ATGAGCTTAGTTCCTTCTGC-3′ (SEQ ID NO: 6). Samples (5 μl) were electrophoresed in 2% agarose gels and visualized by ethidium bromide staining. To analyze BCR rearrangements, PCR was performed to detect rearranged VH genes using a mixture of forward primers with a single reverse primer as previously described.

### H&E Staining, Immunohistochemistry (IHC)

H&E staining was done as per established protocols. For IHC, formalin-fixed, paraffin-embedded (FFPE) tissue sections were deparaffinized with xylene and hydrated, and antigen retrieval was done in Tris-EDTA buffer (pH 9.0). The slides were boiled in the antigen retrieval buffer for 20 minutes, cooled for 40 minutes at room temperature, then washed once in 1×TBS with 0.05% Tween-20 (pH 7.6). Blocking was done at room temperature for 2 h with 10% FBS. Sections then were incubated in primary antibody overnight at 4° C. using manufacturer's recommended concentrations; Ki-67 (Santa Cruz Biotechnology, sc-23900), pATR (S428) (Cell Signaling Technology, 2853), ATR (Bethyl Laboratories, Inc., A300-137A and A300-138A), CD3e (Thermo Fisher Scientific, MA1-7630), CD5 (Thermo Fisher Scientific, MA5-13308), CEA (Thermo Fisher Scientific, 14-0661-82), Melan A (Proteintech, 18472-1-AP), CD20 (Thermo Fisher Scientific, MA1-7634), CD19 Thermo Fisher Scientific, 14-0194-82), AFP (Proteintech, 14550-1-AP). Sections were washed once, and endogenous peroxidase was quenched using 0.3% hydrogen peroxide for 15 minutes at room temperature. After washing the sections, secondary antibody incubation was done using the appropriate biotinylated secondary antibodies; goat anti-rabbit (Sigma Aldrich, SAB3700880), and goat anti-mouse (Sigma Aldrich, SAB3701075). Sections then were developed using DAB (Sigma Aldrich, D4293). All sections were counterstained with Harris-modified hematoxylin solution (Millipore Sigma, HHS32).

All microscopic images were acquired with an Olympus VS120 Slide Scanner Slide Analyzer and statistical analyses between control and tumor sections were conducted with the two-tailed Student's t-test.

### Mouse Embryo Collection

Female mice (10-12 weeks) were super-ovulated by injection with 7-10 IU pregnant-mares serum gonadotrophin (PMSG) (Bioworld, 22060640-1). After 48 h, an injection of 7-10 IU human chorionic gonadotrophin (hCG) (Sigma Aldrich, C1063) was administered before mating with a male mouse of the same species. Successful matings were assessed by the presence of a vaginal sperm plug the following morning. Mouse embryos were collected between days 3-14 post-hCG as indicated by the experimental protocol. At collection, female reproductive tracts were dissected out and oviducts and uteri were flushed with M2 embryo media (Sigma-Aldrich, M7167). Flushed and collected embryos were washed and were either snap-frozen and stored at −80° C., to be used for further analysis, or fixed in 2% paraformaldehyde for H&E staining.

### Animal Procedures

Mice were maintained and tissues were collected for research purposes under protocols approved by the University of Toledo's and by East Tennessee State University's Animal Use and Care Committees (IACUC).

### Statistical Analysis

The gene expression data of Cancer Genome Atlas (TCGA) database was downloaded from the Human Protein Atlas27 website (https://www.proteinatlas.org/) in January 2021, where data has been cleaned and mRNA expression data in FPKM have been matched to the demographic characteristics data for each patient. The overall survival followed up to 1825 days were analyzed using the Kaplan-Meier method and the log-rank test. Cox proportional hazard (Cox PH) models were employed with Hazard Ratios (HR) to quantify the magnitude and direction of the association analysis. The potential confounding factors including age, race, gender, and tumor stage upon diagnosis were tested using Cox PH models and the factors that have significance were included in the multivariable regression analyses. The proportional hazard assumption was tested by examining scaled Schoenfeld residuals with p-values adjusted using Bonferroni's correction. All p-values are reported corresponding to two-tailed tests with p-value <0.05 to be considered statistically significant. All statistical analyses were done in R version 4.0.3 (https://www.R-project.org/) using Survival package (https://CRAN.R-project.org/package=survival)28 on a Windows platform.

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

1. A method for treating a cancer comprising administering to a subject having a cancer an effective amount of a cis-ATR antagonist or inhibitor together with a cancer therapeutic drug to treat the cancer.

2. The method of claim 1, wherein the cis-ATR antagonist or inhibitor is a PP2A inhibitor.

3. The method of claim 1, wherein the cancer therapeutic drug comprises a chemotherapeutic agent, an immunotherapeutic agent, or a hormonal therapeutic agent.

4. The method of claim 1, wherein the cis-ATR antagonist or inhibitor and the cancer therapeutic drug are administered simultaneously.

5. The method of claim 1, wherein the cis-ATR antagonist or inhibitor and the cancer therapeutic drug are administered sequentially.

6-23. (canceled)

24. A method for treating a cancer comprising administering to a subject having a cancer an effective amount of a cis-ATR antagonist or inhibitor to treat a DNA damaging drug resistant cancer, wherein the cis-ATR antagonist or inhibitor is administered without an additional cancer therapeutic drug.

25. The method of claim 24, wherein the cis-ATR antagonist or inhibitor is a PP2A inhibitor.

26. (canceled)

27. The method of claim 1, wherein the cancer therapeutic drug is selected from the group consisting of: erlotinib, docetaxel, fluorouracil, 5-fluorouracil, gemcitabine, PD-0325901, cisplatin, carboplatin, paclitaxel, temozolomide, tamoxifen, doxorubicin, Akti-1/2, HPPD, rapamycin, lapatinib, oxaliplatin, bortezomib, sutent, letrozole, imatinib mesylate, XL-518, ARRY-886, SF-1126, BEZ-235, XL-147, ABT-869, ABT-263, PTK787/ZK 222584, fulvestrant, leucovorin, lonafamib, sorafenib, gefitinib, irinotecan, tipifamib, capecitabine, abraxane, albumin-engineered nanoparticle formulations of paclitaxel, vandetanib, chloranmbucil, AG1478, AG1571, temsirolimus, pazopanib, canfosfamide, thioTepa and cyclosphosphamide, bullatacin, bullatacinone, bryostatin, callystatin, CC-1065 or analogs thereof, cryptophycin 1, cryptophycin 8, dolastatin, duocarmycin or analogs thereof, leutherobin, pancratistatin, sarcodictyin, spongistatin, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimnustine, clodronate, esperamicin, chromoprotein enediyne antibiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin, epirubicin, esorubicin, idarubicin, marcellomycin, mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin, methotrexate, denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mitoxantrone, mopidanmol, nitraerine, pentostatin, phenamet, pirarubicin, losoxantrone, podophyllinic acid, 2-ethylhydrazide, procarbazine, a polysaccharide complex, razoxane, rhizoxin, sizofuran, spirogermanium, tenuazonic acid, triaziquone, 2,2′,2″-trichlorotriethylamine, T-2 toxin, verracurin A, roridin A, anguidine, urethane, vindesine, dacarbazine, mannomustine, mitobronitol, mitolactol, pipobroman, gacytosine, arabinoside, cyclophosphamide, thioTepa, 6-thioguanine, mercaptopurine, vinblastine, etoposide, ifosfamide, mitoxantrone, vincristine, vinorelbine, novantrone, teniposide, edatrexate, daunomycin, aminopterin, ibandronate, CPT-11, topoisomerase inhibitor RFS 2000, difluoromethylomithine (DMFO), paclitaxel, abraxane, afinitor, erlotinib hydrochloride, everolimus, gemcitabine hydrochloride, oxaliplatin, capecitabine, cisplatin, irinotecan, colinic acid, folfox, folfirinox, nab-paclitaxel with gemcitabine, metformin, digoxin, simvastatin, nivolumab, pembrolizumab, rituximab, durvalumab, cemiplimab, anastrozole, exemestane, letrozole, tamoxifen, raloxifene, fulvestrant, toremifene, gosrelin, leuprolide, triptorelin, apalutamide, enzalutamide, darolutamide, bicalutamide, flutamide, nilutamide, abiraterone, ketoconazole, degarelix, medroxyprogesterone acetate, megestrol acetate, mitotane, and combinations thereof.

28-33. (canceled)

34. A kit comprising:
a first container housing a cis-ATR antagonist or inhibitor; and
a second container housing a cancer therapeutic drug.

35. The kit of claim 34, wherein the cis-ATR antagonist or inhibitor is a PP2A inhibitor.

36. The kit of claim 34, wherein the kit further comprises a pharmaceutically acceptable carrier, diluent, or excipient.

37. A transgenic animal comprising a C57BL/6 mouse having a single amino acid substitution of Ser431 of ATR with alanine, wherein the single amino acid substitution silences phosphorylation of ATR at residue 431, resulting in isomerization of trans-ATR to cis-ATR.

38. (canceled)

39. The method of claim 25, wherein the PP2A inhibitor is LB-100.

40. The method of claim 39, wherein the effective amount is 5 μM.

41. The kit of claim 35, wherein the PPA inhibitor is LB-100.

42. The kit of claim 34, wherein the cancer therapeutic drug is selected from the group consisting of: erlotinib, docetaxel, fluorouracil, 5-fluorouracil, gemcitabine, PD-0325901, cisplatin, carboplatin, paclitaxel, temozolomide, tamoxifen, doxorubicin, Akti-1/2, HPPD, rapamycin, lapatinib, oxaliplatin, bortezomib, sutent, letrozole, imatinib mesylate, XL-518, ARRY-886, SF-1126, BEZ-235, XL-147, ABT-869, ABT-263, PTK787/ZK 222584, fulvestrant, leucovorin, lonafamib, sorafenib, gefitinib, irinotecan, tipifamib, capecitabine, abraxane, albumin-engineered nanoparticle formulations of paclitaxel, vandetanib, chloranmbucil, AG1478, AG1571, temsirolimus, pazopanib, canfosfamide, thioTepa and cyclosphosphamide, bullatacin, bullatacinone, bryostatin, callystatin, CC-1065 or analogs thereof, cryptophycin 1, cryptophycin 8, dolastatin, duocarmycin or analogs thereof, leutherobin, pancratistatin, sarcodictyin, spongistatin, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimnustine, clodronate, esperamicin, chromoprotein enediyne antibiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin, epirubicin, esorubicin, idarubicin, marcellomycin, mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin, methotrexate, denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mitoxantrone, mopidanmol, nitraerine, pentostatin, phenamet, pirarubicin, losoxantrone, podophyllinic acid, 2-ethylhydrazide, procarbazine, a polysaccharide complex, razoxane, rhizoxin, sizofuran, spirogermanium, tenuazonic acid, triaziquone, 2,2′,2″-trichlorotriethylamine, T-2 toxin, verracurin A, roridin A, anguidine, urethane, vindesine, dacarbazine, mannomustine, mitobronitol, mitolactol, pipobroman, gacytosine, arabinoside, cyclophosphamide, thioTepa, 6-thioguanine, mercaptopurine, vinblastine, etoposide, ifosfamide, mitoxantrone, vincristine, vinorelbine, novantrone, teniposide, edatrexate, daunomycin, aminopterin, ibandronate, CPT-11, topoisomerase inhibitor RFS 2000, difluoromethylomithine (DMFO), paclitaxel, abraxane, afinitor, erlotinib hydrochloride, everolimus, gemcitabine hydrochloride, oxaliplatin, capecitabine, cisplatin, irinotecan, colinic acid, folfox, folfirinox, nab-paclitaxel with gemcitabine, metformin, digoxin, simvastatin, nivolumab, pembrolizumab, rituximab, durvalumab, cemiplimab, anastrozole, exemestane, letrozole, tamoxifen, raloxifene, fulvestrant, toremifene, gosrelin, leuprolide, triptorelin, apalutamide, enzalutamide, darolutamide, bicalutamide, flutamide, nilutamide, abiraterone, ketoconazole, degarelix, medroxyprogesterone acetate, megestrol acetate, mitotane, and combinations thereof.

43. The method of claim 1, wherein the PP2A inhibitor is LB-100 and the cancer therapeutic drug is gemcitabine or folfirinox.

44. The method of claim 43, wherein the cancer is pancreatic cancer.

45. The method of claim 2, wherein the PP2A inhibitor is LB-100.


SEQUENCE LISTING (20 sequences)

SEQ ID NO: 1 - DNA, 22 bp, synthetic construct
  misc_feature 1..22: note = Description of Artificial Sequence: Synthetic primer
  source 1..22: mol_type = other DNA
gactcatgtaacacctcatgca

SEQ ID NO: 2 - DNA, 21 bp, synthetic construct
  misc_feature 1..21: note = Description of Artificial Sequence: Synthetic primer
  source 1..21: mol_type = other DNA
acccaaattaaacaggcatgc

SEQ ID NO: 3 - DNA, 24 bp, synthetic construct
  misc_feature 1..24: note = Description of Artificial Sequence: Synthetic primer
  source 1..24: mol_type = other DNA
gagagtgcgcaaatatcctgtata

SEQ ID NO: 4 - DNA, 20 bp, synthetic construct
  misc_feature 1..20: note = Description of Artificial Sequence: Synthetic primer
  source 1..20: mol_type = other DNA
tgggggaattactacgagct

SEQ ID NO: 5 - DNA, 20 bp, synthetic construct
  misc_feature 1..20: note = Description of Artificial Sequence: Synthetic primer
  source 1..20: mol_type = other DNA
tatgtccttgcaacccctac

SEQ ID NO: 6 - DNA, 20 bp, synthetic construct
  misc_feature 1..20: note = Description of Artificial Sequence: Synthetic primer
  source 1..20: mol_type = other DNA
atgagcttagttccttctgc

SEQ ID NO: 7 - DNA, 21 bp, synthetic construct
  misc_feature 1..21: note = Description of Artificial Sequence: Synthetic primer
  source 1..21: mol_type = other DNA
gtgcaaatatcctgtatagtt

SEQ ID NO: 8 - DNA, 20 bp, synthetic construct
  misc_feature 1..20: note = Description of Artificial Sequence: Synthetic primer
  source 1..20: mol_type = other DNA
acagtagtaggtggcttcac

SEQ ID NO: 9 - DNA, 15 bp, synthetic construct
  misc_feature 1..15: note = Description of Artificial Sequence: Synthetic primer
  source 1..15: mol_type = other DNA
atgaaggcccggaca

SEQ ID NO: 10 - DNA, 20 bp, synthetic construct
  misc_feature 1..20: note = Description of Artificial Sequence: Synthetic primer
  source 1..20: mol_type = other DNA
acaagtgttcagaagcccga

SEQ ID NO: 11 - DNA, 20 bp, synthetic construct
  misc_feature 1..20: note = Description of Artificial Sequence: Synthetic primer
  source 1..20: mol_type = other DNA
tggatatctcaggatcagct

SEQ ID NO: 12 - DNA, 18 bp, Mus sp.
  source 1..18: mol_type = genomic DNA
gaggtgtcaccaaagagg

SEQ ID NO: 13 - DNA, 18 bp, synthetic construct
  misc_feature 1..18: note = Description of Artificial Sequence: Synthetic oligonucleotide
  source 1..18: mol_type = other DNA
gaggtggcgcctaagagg

SEQ ID NO: 14 - DNA, 18 bp, synthetic construct
  misc_feature 1..18: note = Description of Artificial Sequence: Synthetic oligonucleotide
  source 1..18: mol_type = other DNA
gaggtgagcgctaagagg

SEQ ID NO: 15 - protein, 6 aa, Mus sp.
EVSPKR

SEQ ID NO: 16 - protein, 6 aa, synthetic construct
  REGION 1..6: note = Description of Artificial Sequence: Synthetic peptide
EVAPKR

SEQ ID NO: 17 - DNA, 18 bp, synthetic construct
  misc_feature 1..18: note = Description of Artificial Sequence: Synthetic oligonucleotide
  source 1..18: mol_type = other DNA
gaggtgkcrccwaagagg

SEQ ID NO: 18 - protein, 6 aa, synthetic construct
  REGION 1..6: note = Description of Artificial Sequence: Synthetic peptide
  MOD_RES 3: note = Ala or Ser
EVXPKR

SEQ ID NO: 19 - DNA, 18 bp, synthetic construct
  misc_feature 1..18: note = Description of Artificial Sequence: Synthetic oligonucleotide
  source 1..18: mol_type = other DNA
gaggtgwsmscwaagagg

SEQ ID NO: 20 - protein, 6 aa, synthetic construct
  REGION 1..6: note = Description of Artificial Sequence: Synthetic peptide
  MOD_RES 4: note = Pro or Ala
EVSXKR